(19) United States
(12) Patent Application Publication
(10) Pub. No.: US 2012/0289887 A1
(43) Pub. Date: Nov. 15, 2012

(54) ELECTROTRANSPORT DEVICES, METHODS AND DRUG ELECTRODE ASSEMBLIES

(75) Inventors: Steven J. Visco, Berkeley, CA (US); Yevgeniy S. Nimon, Danville, CA (US); Bruce Katz, Moraga, CA (US)

(73) Assignee: PolyPlus Battery Company, Berkeley, CA (US)

(21) Appl. No.: 13/556,073

(22) Filed: Jul. 23, 2012

Related U.S. Application Data
(63) Continuation of application No. 12/163,821, filed on Jun. 27, 2008.
(60) Provisional application No. 60/937,709, filed on Jun. 29, 2007.
(60) Provisional application No. 60/970,896, filed on Sep. 7, 2007.
(60) Provisional application No. 61/056,794, filed on May 28, 2008.

(51) Int. Cl. A61N 1/30

(52) U.S. Cl. 604/20; 604/501

(57) ABSTRACT

A drug electrode assembly usefully employed in an electrotransport device for the delivery of drugs across a tissue surface includes an electrode, a drug reservoir which stores the drug (including an ionized (e.g., anionic) or neutrally charged drug species), and a liquid impermeable solid-state assist ion conducting barrier layer interposed between the electrode and the drug reservoir. The barrier layer can be a single-ion conductor of a specific (unique) species of ion called the assist ion. During drug delivery, the assist ion moves across the barrier layer into or out of the drug reservoir, and as the assist ion crosses the barrier layer/drug reservoir interphase, the drug species moves to the tissue surface. The assist ion can be, for example, sodium ions (Na⁺), and the electrode can be an electrode of the assist ion (i.e., a sodium electrode).

### CROSS-REFERENCE TO RELATED APPLICATIONS

This application claims priority to U.S. patent application Ser. No. 12/163,821 filed Jun. 27, 2008, titled ELECTROTRANSPORT DEVICES, METHODS AND DRUG ELECTRODE ASSEMBLIES, now pending, which claims priority to U.S. Provisional Patent Application No. 60/937,709 filed Jun. 29, 2007, titled BIOELECTRODE ASSEMBLIES, DEVICES AND METHODS FOR ELECTRO-TRANSPORT DELIVERY OF DRUGS; and U.S. Provisional Patent Application No. 60/970,896 filed Sep. 7, 2007, titled BIOELECTRODE ASSEMBLIES, DEVICES AND METHODS FOR ELECTRO-TRANSPORT DRUG DELIVERY and U.S. Provisional Patent Application No. 61/056,794 filed May 28, 2008, titled ELECTROTRANSPORT DEVICES, METHODS AND DRUG ELECTRODE ASSEMBLIES. Each of these prior applications is incorporated herein by reference in their entireties and for all purposes.

### FIELD OF THE INVENTION

This invention relates generally to electrotransport drug delivery, and more particularly to drug electrode assemblies and electrotransport devices and methods of administering a drug across a tissue surface of a subject.

### BACKGROUND OF THE INVENTION

Electrotransport drug delivery is generally plagued with poor efficiency, caused in part by competitive ion effects, and electrode degradation which begins immediately upon device activation. Drug delivery can also lead to an ingression of undesired ions into the body circulatory system. Device performance and patient satisfaction are dampened by poor efficiency that necessitates the use of large currents to deliver a therapeutic dose.

Problems with iontophoretic drug delivery are especially prevalent for the delivery of anionic drugs. Generally, if stainless steel polarizing electrodes are employed, pH changes take place in the drug reservoir as a consequence of water electrolysis. These pH changes de-stabilize the drug and are irritating to the skin.

Alternatively, sacrificial electrodes such as Ag/AgCl can be used. Sacrificial positive electrodes, such as chloridized silver, undergo decomposition to give silver metal and a chloride anion. The efficiency is severely hindered because the chloride anion is free to migrate, along with any anionic drug, into the body.

Alternative electrode materials, particularly intercalation compounds, are generally unstable in contact with aqueous environments, and this has precluded their use in practical iontophoretic drug delivery devices.

### SUMMARY OF THE INVENTION

The present invention pertains to drug electrode assemblies and electrotransport devices for delivering a drug across a tissue surface, and to methods for administering drugs, including ionized (both cationic and anionic) and neutrally charged drugs to or across a tissue surface of a subject for which administration of that drug is intended.

In one aspect the present invention pertains to a drug electrode assembly comprising a solid-state barrier layer that is impermeable to liquids and selectively conductive to a specific (i.e., unique) species of ion, generally referred to herein as the assist ion of the assembly, or more simply the assist ion. In various preferred embodiments, because of its biocompatibility, the assist ion is sodium ion (Na⁺).

The barrier layer of the drug electrode assembly is interposed between an electrode and a drug reservoir, which comprises a drug and an assist ion conducting electrolyte solution, the drug is generally dissolved, suspended, blended or otherwise dispersed in the electrolyte solution. The barrier layer may generally be described as having a first and second major surface. The first surface faces the electrode and the second surface faces the drug reservoir. Material phases adjacent to each surface are sometimes referred to herein as being on the electrode side or on the drug side of the barrier layer. During drug delivery, an electrical current flows through the assembly and in support of that current assist ions electrically migrate across the barrier layer from the electrode side to the drug side, or vice versa from the drug side to the electrode. The direction the assist ions migrate depends on the polarity of the electrode and the charge polarity of the assist ion. When the charge polarity of the assist ion and the polarity of the electrode are of the same sign (i.e., both positive or both negative), the assist ions migrate from the drug side to the electrode side, and when the charge polarity of the assist ion and the polarity of the electrode are of opposite sign (i.e., the electrode is positive and the assist ion is negative, or vice versa), the assist ions migrate from the electrode side to the drug side.

In various embodiments, the drug reservoir, and specifically its electrolyte solution, physically contacts and substantially covers at least a portion of the barrier layer second surface, forming an intimate barrier layer/drug reservoir interface for assist ions to move across during drug delivery. In other embodiments, it is contemplated that additional material layers, generally assist ion conducting, may be interposed between the barrier layer and the drug reservoir to enhance interfacial stability or otherwise improve performance.

In certain embodiments, the first surface of the barrier layer contacts and substantially covers at least a portion of the electrode first surface and assist ions move across an intimate electrode/barrier layer interface, where the assist ions are either absorbed or desorbed by the electrode when it is electrochemically oxidized or reduced during drug delivery.

In other embodiments, an assist ion conducting interlayer electrolyte is interposed between the electrode and the barrier layer, the interlayer contacting and substantially covering at least a portion of the electrode surface, and the interlayer contacting and substantially covering at least a portion of the barrier layer first surface. The interlayer forms an intimate interface with both the barrier layer first surface and the electrode first surface that allows assist ions to electrically migrate across the interface during drug delivery. Generally, the interlayer is or comprises a material layer that positively separates the electrode from contact with the barrier layer. In other embodiments, it is contemplated that additional material layers, generally assist ion conducting, may be interposed between the barrier layer and the interlayer to enhance interfacial stability or otherwise improve performance.

The barrier layer is impermeable to materials with which it comes into contact during manufacture, operation and storage of a device into which the electrode assembly is incorporated. Accordingly, the barrier layer is impermeable to electrolyte solutions of the drug reservoir and electrolyte solutions of the interlayer, where present. The barrier layer is also impermeable to solvents of those electrolyte solutions and impermeable to molecules of those solvents. These materials may be solid or liquid phases and combinations thereof (e.g., gels), and preferably even gaseous phases.

In accordance with the instant invention, in various embodiments: when an electrolyte solution adjacent to and in contact with the barrier layer is aqueous, the barrier layer is impermeable to the aqueous solution and impermeable to water molecules; when the electrolyte solution comprises a non-aqueous solvent (e.g., an organic liquid or an organic polymer), the barrier layer is impermeable to that solvent and the molecules of that solvent; when the electrolyte solution is or comprises a gel, the barrier layer is impermeable to both the solid (e.g., polymer matrix) and liquid phases (e.g., aqueous or organic liquid) of the gel and the molecules of the solid phase (e.g., polymer molecules) and the molecules of the liquid phases (e.g., water molecules or organic liquid molecules); when the electrolyte solution comprises a non-aqueous liquid (e.g., a liquid organic solvent), the barrier layer is impermeable to that non-aqueous liquid and impermeable to the molecules which make-up that liquid, for example when the non-aqueous liquid is an organic liquid, such as a protic or an aprotic organic liquid the barrier layer is impermeable to that liquid and the molecules of that liquid.

In specific embodiments, the barrier layer is further impervious to those materials and substances thereof for which it (the barrier layer) is impermeable. In various embodiments the barrier layer is devoid of liquid phases. In some embodiments, the barrier layer is “dry”.

The drug reservoir is generally exposed, at some point during operation and storage, to the environment about the tissue surface, and this environment generally contains moisture (e.g., from the air), and at least some water molecules may, and generally do, absorb into the drug reservoir, for instance into the electrolyte solution of the drug reservoir. Furthermore, during fabrication of the drug electrode assembly, at least part of that fabrication generally takes place in ambient air conditions which contain moisture (i.e., water vapor) as well as other constituents which may be reactive in contact with the electrode, including oxygen and carbon dioxide. And during that fabrication, at least a portion of the barrier layer—generally its second surface—is exposed directly or indirectly to the ambient air of the manufacturing environment. In certain embodiments, the electrode is chemically incompatible in contact with moisture from the ambient air, and in some embodiments the electrode can be further degraded, by contact with oxygen and carbon dioxide. Accordingly, in various embodiments, the barrier layer is generally fluid impermeable and specifically impermeable to air and various constituents thereof including oxygen, water vapor and carbon dioxide.

In specific embodiments, the barrier layer is an ion species selective conductor of the assist ion, meaning that the barrier layer is a highly selective conductor of the assist ion (e.g., showing at least one, preferably at least two-, more preferably at least three- or four- or more orders of magnitude greater conductivity for the assist ion than for any other ion present in the layer). The barrier layer can be a single-ion conductor of the assist ion. Single-ion conducting barrier layers in accordance with the instant invention have an assist ion transference number of at least 0.95, or at least 0.99, or even at least 0.999. The transference number is defined as the ratio of the assist ion conductivity to the total conductivity of the layer, where the total conductivity includes the electronic conductivity plus the ionic conductivity of all ions of the layer.

The barrier layer comprises one or more inorganic assist ion conducting solid-state electrolyte material(s) which form a continuous inorganic solid-state conductive medium that provides a pathway for assist ions to migrate through and across the barrier layer. In order to achieve the requisite barrier properties of a barrier layer, the solid-state electrolyte material of the barrier layer is impervious to those material phases and substances thereof for which the barrier layer is impermeable. Generally, the solid-state electrolyte material of the barrier layer is impervious to liquid phases (including water and organic liquids), water molecules and organic solvent molecules that are present in the electrolyte solution of the drug reservoir and/or that of the interlayer. Preferably, the solid-state electrolyte material of the barrier layer is further impervious to gas phase fluids, particularly ambient air and constituents thereof, including water vapor, oxygen and carbon dioxide, for reasons as described above.

In various embodiments, the barrier layer comprises the solid-state electrolyte material and additional materials which may be incorporated to facilitate fabrication, or close off through porosity, or otherwise improve the performance of the barrier layer. In some embodiments, the barrier layer essentially consists of its solid-state electrolyte material, for instance in the form of an inorganic monolithic solid-state layer. In other embodiments, the barrier layer is a multilayer composite laminate comprising at least two layers, a first assist ion conducting layer forming the barrier layer first surface, the first layer comprising an impervious solid-state electrolyte material and a second assist ion conducting layer forming the barrier layer second surface, the second layer comprising an impervious solid-state electrolyte material which is the same or different than that of the first layer.

In various embodiments, due to a combination of the barrier layer's conductive and barrier properties, the barrier layer is only permeable to assist ions: the solid-state electrolyte material providing the continuous and impervious medium for assist ion migration across the layer and substantially no other ion; and the barrier layer's liquid impermeability precluding the formation of a continuous liquid phase that would otherwise allow ions of various type to diffuse through the barrier layer.

The electrode of the drug assembly can take a variety of forms. In various embodiments, the electrode is a “reactive electrode” in that it comprises a solid-state electroactive material, which itself is electrochemically reduced and/or oxidized during drug delivery. In certain embodiments, the reactive electrode is an alkali metal electrode that when electrochemically reduced or oxidized, absorbs and/or desorbs an alkali metal ion. In various specific embodiments, the alkali metal electrode is a sodium electrode comprising electroactive sodium, for example, a sodium intercalation material, sodium metal or a sodium alloy (including sodium metal alloys and sodium semi-metal alloys).

In various embodiments, the electrode may adversely react in contact with water, and in some embodiments the electrode is chemically incompatible in contact with water, for instance water of an aqueous solution or that which is derived from ambient air, the moisture content of which provides an unlimited source of water that if not for the barrier layer would diffuse through the assembly where it would eventually contact the electrode. The barrier layer protects the electrode by precluding it from contact with water.

In various embodiments, the interlayer is an assist ion conductor and comprises an electrolyte solution that comprises an electrolyte salt dissolved in a solvent. Generally, the electrolyte salt is a salt of the assist ion (meaning that the salt comprises an assist ion and when the salt dissolves the assist ion exists in the electrolyte solution as a mobile ion). In certain embodiments, the electrolyte solvent is water. In other embodiments, the electrolyte solvent is organic, typically a liquid, e.g., an aprotic liquid organic solvent. In other embodiments, the interlayer is a solid-state inorganic assist ion conductor (e.g., a Na⁺ ion conducting glass). In certain embodiments, an interlayer is present and the interlayer is a gel electrolyte comprising an assist ion conducting electrolyte solution impregnated in a solid phase matrix. In other embodiments the interlayer further comprises a separator material such as a semi-permeable membrane imbibed with an assist ion conducting electrolyte solution, typically a liquid or a gel.

In certain embodiments the drug is an ionized drug species. In some embodiments the ionized drug species is negatively charged (i.e., anionic). In other embodiments, it is cationic (i.e., positively charged). In certain embodiments the drug is a neutrally charged drug species. Generally, the drug species is dissolved, suspended blended or otherwise dispersed in the drug reservoir, typically in the electrolyte solution.

In yet another aspect, the present invention pertains to an electrotransport device for delivering a drug across a tissue surface of a subject. The device comprises the drug electrode assembly of the instant invention electrically coupled to a second electrode assembly. In some embodiments, the second electrode assembly is an indifferent electrode assembly; for example, a Ag/AgCl indifferent electrode assembly. In some embodiments, the second electrode assembly is a second drug electrode assembly, substantially the same or different than the first drug electrode assembly. In certain embodiments, the open circuit galvanic potential difference between the drug electrode assembly and the second electrode assembly is greater than 2V, greater than 2.5 V, or in some embodiments it is about 3V or greater.

Also are provided methods of administering a drug species to a subject. In certain embodiments, drug delivery occurs when an electrical current is supplied to the drug assembly and to the second assembly that is sufficient to cause the drug species to move across the tissue surface. In some embodiments thereof, the electrical current supplied is an alternating electrical current. In some embodiments, drug delivery is initiated by activating a switch that allows electrical current to flow between the drug assembly and the second assembly, the electrical current driven in part or in full by an electromotive force provided by the galvanic potential formed between the drug assembly and the second assembly.

These and other features, including associated device implementations, kits and methods, and advantages of embodiments of various aspects of the present invention will now be described in greater detail with reference to the drawings.

### BRIEF DESCRIPTION OF THE DRAWINGS

In the drawings, identical reference numbers identify similar elements.

FIG. 1A schematically illustrates a cross sectional depiction of a generalized drug electrode assembly in accordance with the present invention. The drug electrode assembly 100a comprises an electrode 102, an assist ion conducting drug reservoir 108 comprising a drug intended for delivery, and a liquid impermeable solid state assist ion conducting barrier layer 106 interposed between the electrode and the drug reservoir. Also shown is an exterior electrical connector 111 electrically coupled to the electrode of the assembly and an optional housing 119 for the assembly.

FIG. 1B schematically illustrates a cross sectional depiction of a generalized drug electrode assembly 100b similar to that depicted in FIG. 1A, and further comprising an assist ion conducting interlayer electrolyte 104.

FIG. 2A schematically illustrates a cross sectional depiction of a drug electrode assembly 200a in accordance with a specific embodiment of the instant invention wherein the assist ion of the assembly is sodium ion (Na⁺). The assembly comprising, from an interior to an exterior: a “reactive” sodium electrode 202a having a first surface; a Na⁺ ion conducting non-aqueous interlayer electrolyte 204a; a liquid impermeable solid-state Na⁺ ion conducting barrier layer 206; and a Na⁻ ion conducting drug reservoir 208 comprising a drug and a Na⁺ ion conducting electrolyte solution. Moreover, the reactive sodium electrode is of the assist ion type, the assist ion of the assembly being sodium ions. Also shown is an exterior electrical connector 111 electrically coupled to the electrode of the assembly and an optional housing 119 for the assembly.

FIG. 2B schematically illustrates a cross sectional depiction of a drug electrode assembly 200b in accordance with a specific embodiment of the instant invention comprising, from an interior to an exterior: a solid-state “reactive” sodium electrode 202b having a first surface; a liquid impermeable solid-state Na⁺ ion conducting barrier layer 206; and a Na⁺ ion conducting drug reservoir 208 comprising a drug and a Na⁺ ion conducting electrolyte solution. Also shown is an exterior electrical connector 111 electrically coupled to the electrode of the assembly and an optional housing 119 for the assembly. The electrode forms an intimate inorganic solid-state interface in contact with the inorganic solid-state conductive medium of the barrier layer. Moreover, the reactive sodium electrode is of the assist ion type, the assist ion being sodium ions.

FIG. 2C schematically illustrates a cross sectional depiction of a drug electrode assembly 200c in accordance with a specific embodiment of the instant invention comprising, from an interior to an exterior: a “reactive” electrode 202c not of the assist type having a first surface; a a Na⁺ ion conducting interlayer electrolyte 204c comprising a a Na⁺ ion conducting electrolyte solution; a liquid impermeable solid-state Na⁺ ion conducting barrier layer 206; and a a Na⁺ ion conducting drug reservoir 208 comprising a drug and a Na⁺ ion conducting electrolyte solution. Also shown is an exterior electrical connector 111 electrically coupled to the electrode of the assembly and an optional housing 119 for the assembly.

FIG. 2D schematically illustrates a cross sectional depiction of a drug electrode assembly 200d in accordance with a specific embodiment of the instant invention comprising, from an interior to an exterior: an “inert” electrode 202d having a first surface; a a Na⁺ ion conducting interlayer electrolyte 204d comprising a a Na⁺ ion conducting electrolyte solution containing electro-active chemical species capable of accepting and/or donating an electron; a liquid impermeable solid-state Na⁺ ion conducting barrier layer 206; and a drug reservoir 208 comprising a drug and a Na⁻ ion conducting electrolyte solution. Also shown is an exterior electrical connector 111 electrically coupled to the electrode of the assembly and an optional housing 119 for the assembly.

FIG. 3A schematically illustrates a cross sectional depiction of an electrotransport device 300a in accordance with a specific embodiment of the instant invention. The device comprises a drug electrode assembly 100a similar to that depicted in FIG. 2A and similar elements are similarly numbered, and a second electrode assembly 360. The second electrode assembly is an indifferent electrode assembly comprising an indifferent electrode 362 and an electrolyte reservoir 368 and an exterior electrical connector 361 electrically coupled to the indifferent electrode. The drug electrode assembly and the indifferent electrode assembly are electrically coupled to each via electrically coupling of their exterior connectors to an electronic control/power supply unit 370.

FIG. 3B schematically illustrates a cross sectional depiction of an electrotransport device 300b in accordance with a specific embodiment of the instant invention. The device comprises a drug electrode assembly 200b similar to that depicted in FIG. 2B and similar elements are similarly numbered, and a second electrode assembly 360. The second electrode assembly is an indifferent electrode assembly comprising an indifferent electrode 362 and an electrolyte reservoir 368 and an exterior electrical connector 361 electrically coupled to the indifferent electrode. The drug electrode assembly and the indifferent electrode assembly are electrically coupled to each via electrically coupling of their exterior connectors to an electronic control/power supply unit 370.

FIG. 3C schematically illustrates a cross sectional depiction of an electrotransport device 300c in accordance with a specific embodiment of the instant invention. The device comprises a drug electrode assembly 200c similar to that depicted in FIG. 2C and similar elements are similarly numbered, and a second electrode assembly 360. The second electrode assembly is an indifferent electrode assembly comprising an indifferent electrode 362 and an electrolyte reservoir 368 and an exterior electrical connector 361 electrically coupled to the indifferent electrode. The drug electrode assembly and the indifferent electrode assembly are electrically coupled to each via electrically coupling of their exterior connectors to an electronic control/power supply unit 370.

FIG. 3D schematically illustrates a cross sectional depiction of an electrotransport device 300d in accordance with a specific embodiment of the instant invention. The device comprises a drug electrode assembly 200d similar to that depicted in FIG. 2D and similar elements are similarly numbered, and a second electrode assembly 360. The second electrode assembly is an indifferent electrode assembly comprising an indifferent electrode 362 and an electrolyte reservoir 368 and an exterior electrical connector 361 electrically coupled to the indifferent electrode. The drug electrode assembly and the indifferent electrode assembly are electrically coupled to each via electrically coupling of their exterior connectors to an electronic control/power supply unit 370.

FIG. 4A schematically illustrates a cross sectional depiction of an electrotransport device 400a in accordance with a specific embodiment of the instant invention. The device comprises a drug electrode assembly 200(a,c,d) similar to that depicted in FIGS. 2A, 2C and 2D and similar elements are similarly numbered, and a second electrode assembly 410a. The second electrode assembly is a second drug electrode. In various embodiments, the second drug electrode assembly is similar to the drug electrode assembly 200(a,c,d) depicted in FIGS. 2A, 2C and 2D. Also shown is an exterior electrical connector 111 electrically coupled to the electrode of the assembly and an optional housing 119 for the assembly.

FIG. 4B schematically illustrates a cross sectional depiction of an electrotransport device 400b in accordance with a specific embodiment of the instant invention. The device comprises a drug electrode assembly 200b similar to that depicted in FIG. 2B and similar elements are similarly numbered, and a second electrode assembly 410b. The second electrode assembly is a second drug electrode assembly similar to the first drug electrode assembly. Also shown is an exterior electrical connector 111 electrically coupled to the electrode of the assembly and an optional housing 119 for the assembly.

### DETAILED DESCRIPTION OF SPECIFIC EMBODIMENTS

Introduction

The methods, electrode assemblies and devices of the present invention provide efficient, convenient, safe and cost-effective alternatives to oral and intravenous drug treatments and generally improve electro-transport drug delivery processes. The invention is now described in further detail with respect to specific embodiments that are not meant to limit the invention but to illustrate its features. While a number of the embodiments are generally described in reference to the delivery of drugs in anionic form and for devices that deliver one drug at a time, the present invention is generally suitable for the delivery of drugs in cationic form as well as net neutrally charged drug species, and for the simultaneous delivery of more than one drug at a time from a single device.

There are presently known a wide variety of electrotransport devices; see for example: U.S. Pat. No. 4,250,878 to Jacobsen et al., U.S. Pat. No. 4,474,570 to Ariura et al.; U.S. Pat. No. 5,618,265 to Myers; International Application Pub. No. WO 2007/041323 to Smith. Exemplary electrotransport devices are disclosed in U.S. Pat. No. 6,289,241 to Phipps; U.S. Pat. Nos. 4,744,787 and 7,212,853 to Phipps et al.; U.S. Pat. No. 4,752,285 to Petelenz et al.; and U.S. Pat. Nos. 5,647,844 and 4,927,408 to Haak et al., which demonstrate basic principles of manufacture, operation and performance that may be applicable to implementation of certain embodiments of the present invention given the description that follows. Each of these documents is incorporated herein by reference for the purpose of those relevant teachings.

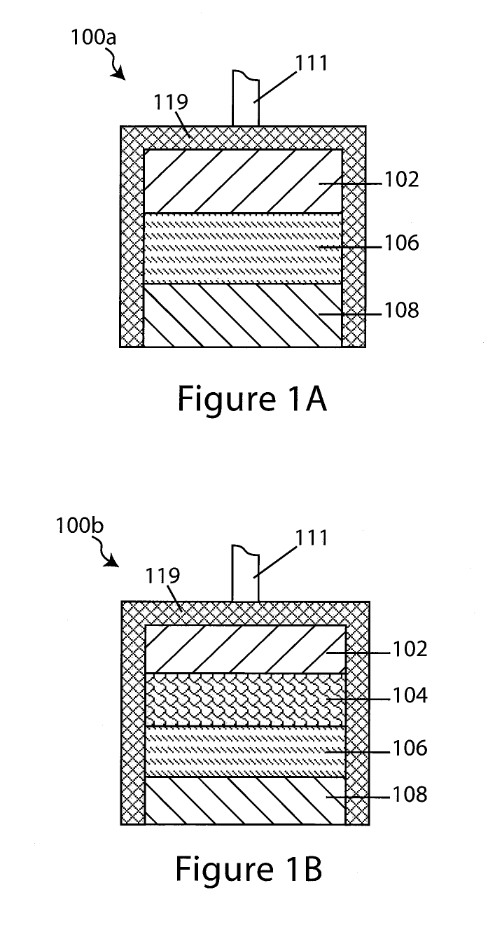

As illustrated in FIG. 1A, a drug electrode assembly in accordance with the present invention comprises an electrode 102; an assist ion conductive drug reservoir 106, which stores a drug; and a liquid impermeable assist ion conducting solid-state barrier layer 106 interposed between the electrode and the drug reservoir. The drug electrode assembly may further comprise an assist ion conducting interlayer electrolyte 104, interposed between the barrier layer and the drug reservoir, as depicted in FIG. 1B.

The barrier layer can be described, generally, as having a first and second surface. The first surface faces the electrode and the second surface faces the drug reservoir, and material phases adjacent to each surface are sometimes referred to herein as being on the electrode side or the drug side of the barrier layer.

The liquid impermeable barrier layer prevents the transmission of liquid and solid phases with which it comes into contact during assembly operation and storage, preventing any such phases from moving between the drug side and the electrode side, while concomitantly providing a continuous solid-state conductive medium for assist ions to electrically migrate through during drug delivery. The barrier layer is also impermeable to molecules of the liquids and solids for which it is impermeable. Preferably the barrier-layer is further impermeable to gaseous phases, such as ambient air, including oxygen, carbon dioxide and water vapor. More preferably, the barrier layer is impervious to those fluids (liquids and gases) to which it is impermeable.

The electrode can take a variety of forms:

In certain embodiments the electrode is a “reactive” electrode. As used herein the term “reactive” electrode refers to an electrode that is itself electrochemically reduced or oxidized. A “reactive” electrode generally comprises a solid-state electroactive component material that is electro-reduced or electro-oxidized during drug delivery, and in the electrochemical process the electroactive component material maintains electrical charge neutrality by absorbing or desorbing charge-compensating ions from or into an adjacent electrolyte phase (e.g., an interlayer electrolyte or the barrier layer). In some embodiments, the electrode is a “reactive” electrode of the assist ion, and by this it is meant that the charge-compensating ions are the assist ion. In other embodiments, the reactive electrode is not of the assist ion, and by this it is meant that the charge-compensating ions are not the assist ion.

In certain embodiments the reactive electrode is an alkali metal electrode comprising a solid-state electroactive alkali metal material, including alkali metals, alkali metal alloys, and alkali metal intercalation materials. In particular embodiments, the alkali metal “reactive” electrode is a sodium “reactive” electrode, comprising a solid-state electroactive sodium material, including sodium metal, sodium alloys, and sodium intercalation materials. In some embodiments the alkali metal “reactive” electrode, e.g., a sodium “reactive” electrode, is of the assist ion.

In some embodiments, the electrode is an “aqueous compatible” “reactive” electrode, including aqueous compatible metal electrodes that plate the metal of the electrode when electro-reduced and strip the metal when electro-oxidized, e.g., Ag, Cu, Fe and Zn metal electrodes, and aqueous compatible metal/metal salt electrodes, e.g., Ag/AgCl, Fe/FeCl, and Cu/CuCl.

In other embodiments, “aqueous incompatible” electrodes and electrodes which are chemically incompatible in contact with water can be used in the assembly of the instant invention because water molecules and for that matter liquid water and aqueous solutions which are present on the drug side cannot permeate through the barrier layer to the electrode side and are therefore prevented from contacting the electrode. For instance, “aqueous incompatible” or for that matter water incompatible alkali metal electrodes are enabled for use herein as an electrode. The “aqueous incompatibility” or water incompatibility of an alkali metal electrode is commonly derived from its capacity to reduce water (e.g., alkali metals reduce water in contact), or in some cases to oxidize it (e.g., certain intercalation electrodes of sufficient electrochemical potential). For instance, alkali metals have an electrochemical potential that is well negative of the reductive decomposition potential of water, and in contact alkali metals reduce water. In fact, the protection provided by the barrier layer generally enables the use of electrodes having an electrochemical potential that is beyond the thermodynamic oxidative or reductive decomposition potential of water, and although such electrodes are thermodynamically unstable in contact with water or for that matter aqueous solutions, they are nevertheless enabled for use herein by virtue of the layer's barrier properties, and in particular its impermeability to water.

The oxidative decomposition of water occurs at an electrode when the potential between the aqueous solution and the electrode is more positive than about 1.23 volts versus a Standard Hydrogen Electrode (hereinafter sometimes referred to as “SHE”) and reductive decomposition occurs at an electrode when the potential between the aqueous solution and the electrode is more negative than −0.83 volts vs. SHE. In accordance with the present invention, electrodes having electrochemical potentials more positive than 1.23 volts versus SHE and more negative than −0.83 volts vs. SHE are enabled for use in the drug electrode assembly of the instant invention, e.g., electrodes having an electrochemical potential versus SHE that is more positive than 1.5 V or 2 V are suitable for use herein as are electrodes that are more negative than −1V, −1.5V, −2V, −2.5V or even more negative than −3V versus SHE.

In fact, the decomposition potential of water is a function of the pH of the aqueous solution. The reductive decomposition of water can be written either as 2H⁺+2e⁻→H₂(g) or, in neutral or alkaline solutions as H₂O+2 e⁻→H₂(g)+2 OH⁻ These two reactions are equivalent and follow the same Nernst equation which, at 25° C. and unit H₂ partial pressure reduces to E=E°−(0.059/2)×2 pH=−0.059 pH. Similarly, the oxidative decomposition of water H₂O→O₂(g)+4H⁺+2 e⁻ is governed by the Nernst equation which similarly becomes E=1.23−0.059 pH. In accordance with the Nernst equation and with various embodiments of the drug electrode assembly of the instant invention, when the drug reservoir comprises an aqueous solution having a pH in the range of about 4 to 10, the electrode may have an electrochemical potential vs. SHE that is more positive than about 1.0 Volt or more negative than about −0.6 Volt; and when the pH of the drug reservoir is in the range of between about 6 to 8, the electrode may have an electrochemical potential vs. SHE that is more positive than about 0.9 Volts or more negative than about −0.5 Volts.

In other embodiments, the electrode is not a reactive electrode, it is an “inert” electrode. As used herein the term “inert” electrode refers to an electrode that is itself not electrochemically reduced or oxidized during drug delivery but rather facilitates electron transfer to or from electroactive chemical species which are present in an adjacent electrolyte phase in physical contact with the inert electrode, and the chemical species in the electrolyte solution are capable of accepting or donating an electron at the applied potential of the electrode.

During drug delivery an electrical current flows through the assembly, and electrochemical reactions take place at the electrode. In some embodiments, the electrode takes the form of a positive electrode (i.e., it is a cathode) during drug delivery and the electrochemical reaction that takes place at or by the electrode is reduction. In other embodiments, the electrode takes the form of a negative electrode (i.e., it is an anode) during drug delivery and the electrochemical reaction that takes place at or by the electrode is reduction. In certain embodiments, the electrode switches between taking the form of a positive and a negative electrode during the course of drug delivery, e.g., when an alternating current is supplied to the drug electrode assembly.

In certain embodiments an interlayer electrolyte is interposed between the electrode and the barrier-layer, and may provide a positive separation that prevents the electrode from contact with the barrier-layer. In some embodiments, when the electrode is “aqueous compatible”, the interlayer is aqueous and comprises an aqueous electrolyte solution. In other embodiments, the interlayer is non-aqueous. In some embodiments the non-aqueous interlayer electrolyte is an inorganic solid-state assist ion conductor. In other embodiments, the interlayer comprises a non-aqueous electrolyte solution, typically an assist ion conducting liquid organic electrolyte solution, imbibed in a material layer or combined with a solid phase material (e.g., a matrix material) to form a gel electrolyte, typically a polymeric gel electrolyte.

The drug reservoir stores the drug and conducts assist ions. During drug delivery assist-ions move across the barrier-layer and into or out of the drug reservoir. Generally, the drug reservoir comprises an assist-ion conducting electrolyte solution. The drug can be negatively, positively or neutrally charged.

In certain embodiments the drug reservoir is formulated to minimize competitive ion effects when delivering an ionic drug. By one expedient competitive ion effects are minimized by incorporating drug ions and assist ions into the electrolyte solution via dissolution of a drug salt of the assist ion, which is a salt that when dissolved brings to bear mobile drug ions and mobile assist ions in the electrolyte solution. For instance, when the drug ion is an anion and the assist ion is a cation, e.g., Na+ ion, the drug salt may be a sodium drug salt.

In certain embodiments, the drug reservoir is formulated to optimize biocompatibility when delivering a drug species using an alternating current whereby the polarity of the electrode alternatively switches between taking the form of a positive and a negative electrode. In this embodiment, the electrolyte solution of the drug reservoir is formulated by dissolving a biocompatible assist ion salt that when dissolved in the electrolyte solution brings to bear mobile assist ions and mobile counter ions that are preferably biocompatible in contact with the tissue. For instance, when the assist ion is sodium ion, the mobile counter ion can be chlorine ion, and the electrolyte solution formed by dissolving NaCl salt in water.

In accordance with the present invention there is provided a drug electrode assembly having a liquid impermeable assist ion conducting solid-state barrier layer that positively separates the electrode from the drug reservoir, which stores the drug. The barrier layer also protects the electrode from contact with substances which are in the drug reservoir, or for that matter about the tissue surface (e.g., ambient air), that if not for the barrier layer would diffuse through the assembly and adversely react with the electrode. The barrier layer provides further protection in that it prevents various substances, which may be present on the electrode side of the barrier layer, such as the interlayer electrolyte and substances thereof, from reaching the drug reservoir, or even coming into contact with the tissue surface. The barrier layer provides this positive separation and protection while simultaneously providing a continuous solid-state medium for the electrical migration of a specific and unique species of ion (generally referred to herein as the assist ion species) to move across the barrier layer during drug delivery. The assist ion species, or more simply the assist ions, electrically migrate, under the influence of an electrical field, across the barrier layer into or out of the drug reservoir when current flows through the assembly during drug delivery, as drug species move to the tissue.

Generally, the assist ion facilitates drug delivery by electrically migrating across the barrier layer, from the drug side to the electrode side or vice versa from the electrode side to the drug, when an electrical current flows through the assembly. Without intending to be limited by theory, in one embodiment for the delivery of ionized drugs, the assist ion may facilitate drug delivery by electrically migrating across the barrier layer into or out of the drug reservoir in order to maintain charge neutrality conditions within the drug reservoir as the drug ion leaves the reservoir on its way the tissue. And again without limitation to theory, the assist ion may facilitate drug delivery by electrically migrating out of the drug reservoir and across the tissue surface, dragging drug species, e.g., neutrally charged drug species, along with it via electroosmosis, or the electrical migration of the assist ion conditions the tissue as it crosses the tissue surface, perhaps even creating electropores, and by this expedient enhances drug diffusivity, or some combination of electroosmosis and surface conditioning.

The assist ion is generally a monovalent simple ion, and typically it is a monovalent simple cation. A simple ion is defined as an ion formed from a single atom. Suitable “assist ions” include certain transition metal simple ions (e.g., Cu⁺ and Ag⁺), certain halide simple ions (e.g., F− and Cl− ) and alkali metal simple ions (e.g., Li⁺, Na⁺, K⁺), particularly Na⁺ ions. Sodium ion (Na⁻) is a distinctly appealing assist ion because of its exceptional biocompatibility and the fact that sodium ion is the primary cation in extracellular fluids (including blood plasma) in animals and humans. And in the case of anionic drug delivery, the widespread availability of and pharmacologic familiarity with drugs formulated as a sodium salt provides a further advantage as dissolution of the sodium drug salt into the drug reservoir provides both anionic drug species and Na⁺ assist ions.

The barrier layer has both conductive and barrier properties. The barrier layer provides a continuous solid-state assist ion conductive medium, the ionic conductivity of which is highly species specific so that during drug delivery assist ions permeate through the solid-state conductive medium and substantially no other ion. Moreover, the barrier layer's liquid impermeability precludes the formation of a continuous liquid phase that would otherwise provide a medium for ions, generally of any type, to diffuse or otherwise move through the barrier layer. Accordingly, in various embodiments, only the “assist ions” move across the barrier layer during drug delivery and substantially no other ions, for example, only Na⁺ ions (when it is the assist ion) and substantially no other ions. And by this expedient, extraneous ions (i.e., ion species other than that of the assist ion species) generated at the electrode, or generally present in the interlayer, are prevented from moving into the drug reservoir where they can compete with the drug species for delivery and/or are generally undesirable in contact with the tissue surface.

Furthermore, the barrier layer provides this continuous solid-state medium for assist ion migration while concomitantly preventing the transmission of material phases (e.g., liquids) and various substances derived thereof from moving through it. And by this expedient, the composition and structure of material phases on one side of the barrier layer can be optimized independent of their chemical stability in contact with material phases on the other side of the layer. And this leads to a number of key advantages including: 1) enabling the use electrodes (e.g., sodium electrodes) that are chemically incompatible in contact with material phases that may be present in the drug reservoir and substances derived thereof, and would otherwise, if not for the barrier layer, adversely react with the electrode and render the electrode inoperable; 2) enabling the use of material phases on the electrode side of the barrier layer, such as an organic liquid electrolyte solution, which is desirably kept from contact with the drug or with the tissue itself (e.g., skin); 3) enhancing delivery efficiency of ionized drugs and improving biocompatibility, because competitive ions which may be present in a material phase on the electrode side of the barrier layer, for example ions generated by the electrode during drug delivery, are prevented from migrating across the barrier layer to the drug reservoir where they can compete with the drug for delivery or otherwise adversely interact with the subject, and therefore there presence in the drug reservoir is generally undesirable, e.g., the presence of the barrier layer mitigates competitive ion effects of a metal/metal-salt electrode without relying on precipitation methods; 4) facilitating electrotransport delivery of drug species, e.g., neutrally charged drug species, using a biocompatible assist ion, for example Na⁺ ions, that may penetrate the tissue surface during drug delivery.

In order to achieve the requisite properties and functionality of a barrier layer, the barrier layer comprises at least one impervious solid-state electrolyte material that conducts assist ions. The impervious solid-state electrolyte material is impervious to those material phases and substances thereof for which the barrier layer is impermeable. And the continuous assist ion conductive medium of the barrier layer is composed of the impervious solid-state electrolyte material(s).

The impervious solid-state electrolyte material of the barrier layer may be an amorphous solid-state inorganic material, such as an inorganic glass, an inorganic crystalline material such as an inorganic polycrystalline ceramic or an inorganic glass-ceramic, having the properties which are consistent with the principles of an impervious solid-state electrolyte material as described above. Accordingly, the continuous inorganic solid-state assist ion conducting medium of the barrier layer may be a glass, ceramic (e.g., an inorganic polycrystalline ceramic), glass-ceramic, or combination thereof.

The barrier layer is composed, in whole or in part, of the impervious solid-state electrolyte material or a combination of such impervious solid-state electrolyte materials. In certain embodiments, the barrier layer is a continuous, monolithic layer of the impervious solid-state electrolyte material (e.g., as a sintered sheet or glass-ceramic plate). The solid-state electrolyte material(s) may be distributed uniformly or discretely throughout the barrier layer. In some embodiments, the barrier layer is simply a compositionally homogenous layer of an impervious solid-state electrolyte material. In other embodiments, the barrier layer is a multi-layer composite laminate of two or more assist ion conducting layers, each layer comprising an impervious solid-state electrolyte material. For instance, a first layer of the composite laminate forming the barrier layer first surface and optimized for its chemical compatibility in contact with constituents on the electrode side of the assembly, and the second layer forming the barrier layer second surface and optimized for its chemical compatibility in contact with constituents on the drug side. In some embodiments the solid-state electrolyte material of the first layer is an assist ion conducting inorganic glass and the impervious solid-state electrolyte material of the second layer is an assist ion conducting inorganic polycrystalline ceramic material. In other embodiments, the solid-state electrolyte material of the first layer is an assist ion conducting inorganic polycrystalline ceramic material and the impervious solid-state electrolyte material of the second layer is an assist ion conducting inorganic glass. In certain embodiments, the multilayer composite laminate comprises a third layer interposed between the first and the second layer. In one embodiment, the third layer comprises a solid state electrolyte material which is an assist ion conducting polycrystalline ceramic and the solid-state electrolyte material of the first and the second layer is an assist ion conducting glass of the same or different glass composition.

During drug delivery an electrical current flows through the assembly, and an electric current (in the form of an electronic current) flows into the assembly from an exterior connector 111 electrically coupled to the electrode (e.g., a wire soldered to the electrode). When the electric current into the assembly is a positive current, electrons flow out of the assembly, from the electrode to the exterior connector, and the electrode takes the form of a negative electrode. A negative electrode behaves as an anode, and electro-oxidation reactions take place at the negative electrode. When the electric current into the assembly is a negative current, electrons flow into the assembly, from the exterior connector to the electrode, and the electrode takes the form of a positive electrode. A positive electrode behaves as a cathode, and electro-reduction reactions take place at the positive electrode.

During drug delivery when the electrical current flows through the assembly, an ionic current, carried by assist ion electrical migration, flows across the barrier layer. The direction the “assist ions” electrically migrate depends on the polarity of the electrode, i.e., whether it takes a positive or negative form and on the charge polarity of the assist ion, i.e., whether the assist ion is negatively charged (i.e., an anion) or positively charged (i.e., a cation). When the electrode polarity and the charge polarity of the assist ion are of the same sign, assist ions electrically migrate across the barrier layer from the drug side to the electrode side. When the electrode polarity and the charge polarity of the assist ion are of opposite sign, assist ions electrically migrate across the barrier layer from the electrode side to the drug side. For instance, when the assist ion is a cation, such as an alkali metal ion, e.g., Na⁺ ion, a positive electrode drives the electrical migration of Na⁺ ions across the barrier layer from the drug side to the electrode side and a negative electrode drives the electrical migration of Na+ ions across the barrier layer from the electrode side to the drug side.

Moreover, in order to support the electrical migration of assist ions across the barrier layer during drug delivery there is needed on one side of the barrier layer a material phase or phases that provides a source having a sufficient amount of assist ions to support the migration of assist ions into the barrier layer, and on the opposing side there is a needed a material phase or phases having a sufficient capacity to cumulate assist ions as they migrate out of the barrier layer, sometimes referred to herein as a sink. On the drug side, the assist ion source and sink is generally the drug reservoir, and particularly it is the electrolyte solution. On the electrode side, the source and the sink is generally either the electrode or the interlayer electrolyte.

In some embodiments, when the electrode is a reactive electrode of the assist ion, the electrode is the source and/or the sink. For instance, when the electrode is a sodium “reactive” electrode that takes a positive polarity during drug delivery, it (the electrode) functions as a sink by absorbing sodium ions when it is electro-reduced (e.g., sodium ions insert into a sodium intercalation or sodium alloy electrode, or sodium ions are reduced and plate onto a sodium metal electrode). Likewise, a sodium “reactive” electrode that takes a negative polarity during drug delivery functions as a source of sodium ions by desorbing sodium ions when it is electro-oxidized (e.g., sodium ions de-intercalate from a sodium intercalation electrode or are de-inserted from a sodium alloy electrode, or are desorbed from a sodium metal electrode via a metal striping reaction).

In other embodiments, when the electrode is not a reactive electrode of the assist ion, the interlayer electrolyte is the source and/or the sink. For instance, when the electrode is a Ag/AgCl electrode and the assist ion is sodium ions, the electrolyte solution of the interlayer provides a sink for which sodium ions cumulate during drug delivery when the electrode takes the form of a positive electrode. The sodium ions may exist in the electrolyte solution in the form of dissociated ions, or the sodium assist ions may form a sodium precipitate in the interlayer, or both may occur. Likewise, when the Ag/AgCl electrode takes the form of a negative electrode the interlayer electrolyte provides a source of sodium assist ions, which generally exist in the electrolyte solution in the form of mobile sodium ions.

In accordance with the instant invention, the source component(s) contain a sufficient amount of assist ions and the sink component(s) contain a sufficient capacity to cumulate assist ions in order to support the electrical current through the assembly, and in particular the electrical migration of assist ions, during drug delivery. Without limitation the assist ion may exist in the source or in the sink as an ion, or as a salt, or as an atom.

In various embodiments, for the delivery of an ionized drug (be it anionic or cationic), the electrical charge of the drug species and that of the assist ion can be of opposite sign: A negative electrode assembly driving cationic drug species and a positive electrode assembly driving anionic drug species to electrically migrate to the tissue surface from the drug reservoir. For instance, during drug delivery electrical charge neutrality of the drug reservoir is maintained as the ionic drug species and the “assist ions” concurrently move out of the drug reservoir—in opposing directions. The ionic drug species electrically migrate to the tissue surface while “assist ions” electrically migrate from the drug reservoir across the barrier layer to the electrode side. For example, when the drug intended for delivery to the subject is anionic, a positive electrode drives the anionic drug species to the tissue surface while the assist ions, for example Na⁺ ions, concurrently electrically migrate across a Na⁺ ion conducting barrier layer from the drug reservoir toward the electrode.

In various embodiments, drug species, including neutrally charged drug species, may also be delivered to the subject, facilitated by electrical migration of ionic species through the tissue surface. For instance, ions—particularly small cations (e.g., Na⁺ ions)—can electrically migrate through tissue and in the process pierce the tissue surface, thereby forming electropores that may facilitate diffusion of neutral drug species; or the ions may drag the neutral drug species along with them as they move through the drug reservoir and then penetrate across the tissue surface, for instance via electroosmosis; or the electrical migration of the ions across the tissue surface effectively conditions the tissue for enhanced rate of delivery; or a combination thereof. For instance, when the assist ion is Na⁺ ions a negative electrode may be used to drive Na⁺ ions across the barrier layer, into the drug reservoir and across the tissue surface, which thereby facilitates delivery of the drug specie.

In some particular embodiments, the drug electrode assembly is configured for the delivery of an ionic drug when the drug ion and the assist ion have opposite charge polarity, and during drug delivery the electrode takes on a polarity having the same sign as the charge polarity of the assist ion. For instance, when the assist ion is sodium ions and the ionic drug is a drug anion, the electrode takes the form of a positive electrode.

In other particular embodiment, the drug electrode assembly is configured for the delivery of a charge neutral drug when the electrode takes on a polarity of opposite sign to that of the charge polarity of the assist ion. For instance, a neutral drug species is delivered to the tissue when the assist ion is sodium ions and the electrode takes the form of a negative electrode

In certain particular embodiments, the drug electrode assembly is configured for delivery of a drug species when the electrode is alternatively switched between taking the form of a positive and a negative electrode.

In accordance with the invention there are also provided herein electrotransport drug delivery devices comprising a drug electrode of the instant invention electrically coupled, via an electronic control/power supply unit, to a second electrode assembly. In certain embodiments the second electrode assembly is an indifferent electrode assembly, and in other embodiments the second assembly is a second drug electrode assembly. In certain embodiments, the second drug electrode assembly is similar to the inventive drug electrode assembly of the instant invention. And in certain embodiments, the second drug electrode assembly is substantially the same as the first. Moreover, by proper selection of the electrodes in each assembly, a significant galvanic potential can be generated that is sufficient to fully or partially provide the electromotive force to drive the device current.

In various embodiments, during drug delivery a drug is delivered to the tissue of a subject intended for delivery by supplying an external electrical current between the drug electrode assembly and the second assembly. In some embodiments, during drug delivery the drug electrode assembly takes the form of a positive assembly and the second assembly is negative. In other embodiments, during drug delivery the drug electrode assembly takes the form of a negative assembly and the second assembly is positive. In certain embodiments, the drug electrode assembly alternates or alternatively switches between taking the form of a positive and negative assembly, when an alternating electrical current is supplied between the two assemblies or the supplied electrical current flow switches direction. In some embodiments, a significant open circuit galvanic potential difference is generated between the drug electrode assembly and the second assembly. In certain embodiments, the galvanic potential difference provides or augments the electromotive force that drives the drug to the tissue and the assist-ions across the barrier-layer.

Definitions

To facilitate a better understanding of the present invention, rather than to limit its scope, the following definitions are provided:

As used herein the term “chemically incompatible” refers to a material which when in contact with another material undergoes an adverse reaction that renders the reference material inoperable for its intended purpose. A “chemically compatible” material does not undergo such a reaction.

As used herein the term “aqueous compatible” refers to a material, particularly an electrode that is chemically compatible in contact with aqueous solutions.

As used herein the term “aqueous incompatible” refers to a material, particularly an electrode that is chemically incompatible in contact with aqueous solutions.

As used herein the term “assist ion” is meant to refer to a specific (i.e., unique) species of ion for which a barrier layer is conductive. Assist ions move across the barrier layer from the electrode side to the drug side or vice versa from the drug side to the electrode, during drug delivery. The direction the assist ions move depends on their charge polarity, be it a cation or anion, and whether the electrode of the assembly takes the form of a negative or a positive assembly during drug delivery. When referring to the assist ion in various components of a drug electrode assembly, the assist ion in each of those components is the same assist ion, and as such is sometimes referred to herein as the assist ion of the assembly.

As used herein the term “reactive” electrode means an electrode that itself undergoes electrochemical oxidation and reduction and maintains electrical charge neutrality by absorbing or desorbing an ion of appropriate charge polarity. More specifically, “reactive” electrodes are or comprise an electroactive material which is that portion of the “reactive” electrode that itself undergoes electrochemical oxidation and reduction and maintains electrical charge neutrality by absorbing or desorbing an ion of appropriate charge polarity.

As used herein the term “electrotransport” refers to methods, assemblies, devices and apparatus for delivery of a biologically beneficial agent, whether charged or uncharged, by means of an electromotive force to a drug reservoir containing the biologically beneficial agent. Electrotransport processes include without limitation the delivery of charged drug specie(s) by electromigration, the delivery of uncharged drug specie(s) via electro-osmosis, the delivery of drug specie(s) via electroporation, and the delivery of drug specie(s) by any combination of electromigration, electroosmosis and electroporation.

As used herein the term “electrotransport current” refers to the electrical current which flows through the electrode assembly and which causes electro-oxidation or electro-reduction to occur at the electrode and which brings about electrical migration of assist ions across the barrier layer, and generally also leads to drug delivery to or across the tissue surface.

As used herein the terms “drug,” “drug specie(s)” or “biologically beneficial agent” or more simply “agent” or “agent specie(s)” are used interchangeably and are intended to have their broadest interpretation as any agent that elicits a biological response from a subject being treated with the agent, including the delivery of an agent for purposes other than to treat some condition, for example to facilitate diagnosis. A referenced drug may be completely charged (i.e., 100% ionized), completely uncharged, or partly charged and partly uncharged. As used herein the term anionic drug generally refers to agents that are negatively charged or are partly negatively charged, and the term cationic drug generally refers to agents that are positively charged or are partly positively charged, and the neutral drug generally refers to agents that are uncharged.

As used herein the term “impermeable” generally refers to a material layer which prevents a substance for which it is impermeable from moving across the layer, from one side of the layer to the other side of the layer.

As used herein the term “impervious” refers to a solid-state material that prevents another material phase, be it a liquid, solid or a gas, from penetrating into it.

As used herein the term “drug delivery” refers to the operation of a drug electrode assembly or an electrotransport device and apparatus thereof, whereby an electrical current flows through the drug electrode assembly and assist ions move across the barrier layer. Generally drug is also delivered to the tissue surface when current is flowing. Drug may be delivered passively after current has been stopped.

As used herein, the term “subject” generally refers to any animal, vertebrate or invertebrate, particularly humans.

Drug Electrode Assembly

In various embodiments the preferred assist ion of the drug electrode assembly is Na⁺ ions, and this is so, in part, because Na⁺ ions have exceptional biocompatibility and are generally physiologically native to the subject being treated, sodium is the primary cation in extracellular fluids (including blood plasma) in animals and humans. The invention is now described in more detail with reference to four drug electrode assembly embodiments wherein the assist ion is Na⁺ ions.

The drug electrode assembly in any one of the four embodiments illustrated and described below comprises some features that may be common to all four embodiments, including exterior components such as the housing support structure 119 and the exterior electrical connector 111, as well as assembly innards, specifically the barrier layer 206 and the drug reservoir 208. Accordingly, in order not to obscure the description of each embodiment, similar elements will be numbered similarly and those similar elements are described once, and that description is then referred to thereafter. For the sake of clarity each of the four embodiments are now briefly described with reference to the figures, and a description of more detail follows thereafter.

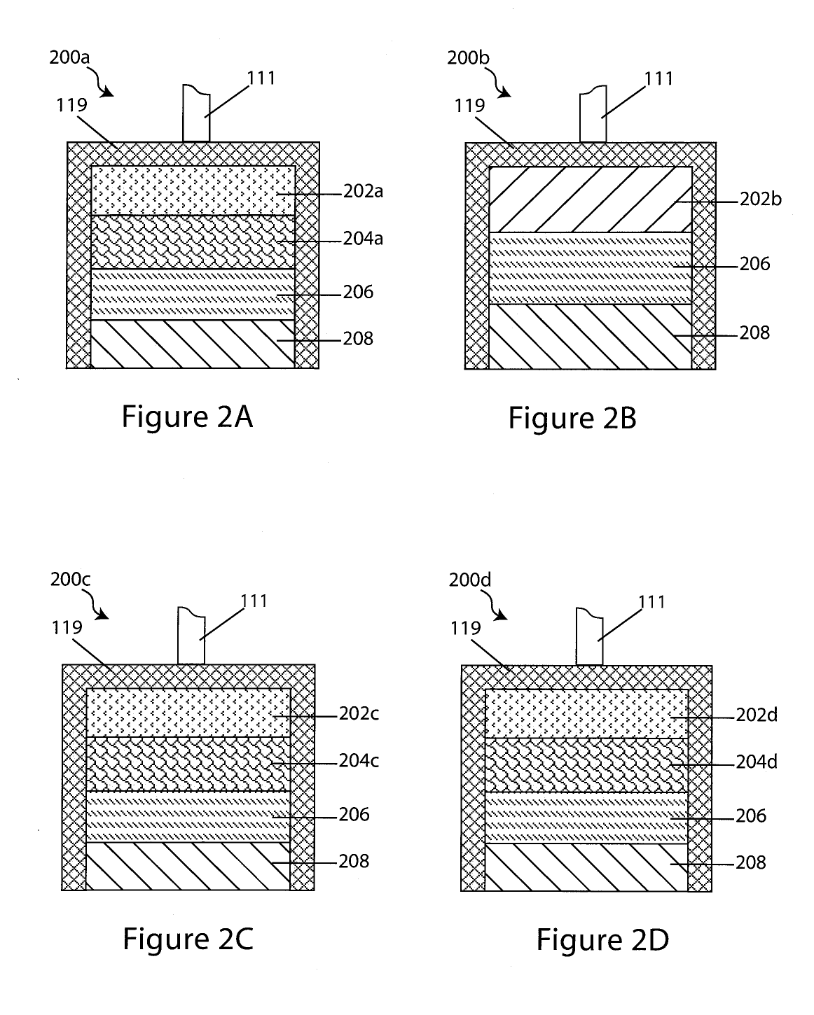

In a first embodiment depicted in FIG. 2A, the drug electrode assembly 200a comprises a Na⁺ ion (i.e., assist ion) conductive non-aqueous interlayer electrolyte 204a interposed between an “aqueous incompatible” sodium “reactive” electrode 202a, and a Na⁺ ion (i.e., assist ion) conducting liquid impermeable solid-state barrier layer 206. The assembly further comprises a drug reservoir 208, configured adjacent to the barrier layer and opposing the interlayer. The drug reservoir 208 stores the drug intended for delivery to the subject, and it is conductive to Na⁺ ions (i.e., the assist ion). Moreover, the “reactive” electrode is of the assist ion, it a sodium electrode and the assist ion of the assembly is sodium ions (Na+ ions).

In a second embodiment depicted in FIG. 2B, the drug electrode assembly 200b comprises an electrode 202b having a first surface, a drug reservoir 208 and a barrier layer 206 interposed between the drug reservoir 208 and the electrode 202b. The barrier layer and the drug reservoir depicted in FIG. 2B are similar to that depicted in FIG. 2A, and similar elements are similarly numbered. In a preferred embodiment, the electrode 202a is a fully solid-state inorganic sodium “reactive” electrode that forms an intimate inorganic solid-state interface with the inorganic solid-state Na⁺ ion conductive medium of the barrier layer 206. Moreover, the “reactive” electrode is of the assist ion, it a sodium electrode and the assist ion of the assembly is sodium ions (Na+ ions).

In a third embodiment depicted in FIG. 2C, the drug electrode assembly 200c comprises an “aqueous compatible” “reactive” electrode 202c that is not of the assist ion. The assembly 200c further comprises a Na⁺ ion conducting interlayer electrolyte 204c comprising a Na⁺ ion conducting electrolyte solution, which is generally aqueous though the invention is not limited as such, interposed between the barrier layer 206 and the electrode 202c. The drug reservoir 208 is as described above, adjacent to the barrier layer and opposite the interlayer 204c. The assist ion of the assembly is Na+ ions.

In a fourth embodiment depicted in FIG. 2D, the drug electrode assembly 200d comprises an “inert” electrode 202d and the interlayer electrolyte 204d comprises a chemical species capable of accepting or donating an electron at the applied potential of the electrode, the interlayer 204d also comprises a Na⁺ ion conducting electrolyte solution, which is generally aqueous though the invention is not limited as such. The assist ion of the assembly is Na+ ions.

With reference to all four embodiments there is depicted in FIGS. 2A-2D, schematically, in cross section, a drug electrode assembly (200a-d) in accordance with the instant invention, that can be usefully employed in electrotransport devices or apparatus' for delivering drugs to a subject across a tissue surface, including the delivery of ionized drugs, both cationic and anionic, as well as neutrally charged drugs. It is to be understood that the drug electrode assembly depicted therein provides but one of at least two electrode assemblies needed for a drug delivery device or apparatus. The other electrode assembly can be a remote assembly or a second assembly of an electrotransport device, and the other assembly can be an indifferent electrode assembly or another drug electrode assembly.

The drug electrode assembly (200a-d) includes a housing support structure 119, generally of an inverted “U” shape and electrically insulating, for instance a molded polymer or otherwise shaped plastic, such as polyvinyl chloride or polyethylene or the like. The housing generally provides protection against the ingression of constituents present in the exterior 199 of the housing, including ambient air. Without limitation, the housing may have a sufficient wall thickness to prevent transmission of air or water vapor, or integrated within or laminated on the housing wall a material layer such as a metal foil or sheet of effective thickness (e.g., Al foil of at least 50 micron thick or thicker) can be used to provide a gas barrier. While the assembly housing may be provided by the housing structure of an electrotransport device, for instance when the assembly is itself incorporated in such a device, generally the drug electrode assembly has its own discrete housing structure.

From an interior 198 to an exterior 199 of the housing, the innards of the drug electrode assembly (200a-d) include an electrode (202a-d) generally in the form of a layer having a first surface; when present, a Na+ ion conducting interlayer electrolyte (200a,c,d), generally referred to herein as the interlayer; a Na⁺ ion conducting liquid impermeable solid-state barrier layer 206 (generally referred to herein as the barrier layer); and a drug reservoir 208, which stores the drug intended for delivery to the subject and conducts Na+ ions. The barrier layer 206 can generally be described as having two major and substantially opposing sides or surfaces. The first surface faces the electrode (202a-d)and the second surface faces the drug reservoir. Material phases adjacent to the first and second surface of the barrier layer are sometimes referred to herein as being on the electrode side or the drug side of the barrier layer. For instance the electrode and the interlayer are on the electrode side and the drug reservoir is on the drug side.

Also there is depicted an exterior electrical connector 111 which in this embodiment is an electronically conducting wire. And when the drug electrode assembly is incorporated into an electrotransport device or apparatus, the exterior connector 111 electrically couples the electrode electrode (202a-d)to an external electronic control/power supply unit (not shown), which is, in turn, electrically coupled to a second electrode assembly (also not shown).

The barrier layer 206, which is interposed between the electrode electrode (202a-d) and the drug reservoir, may be sealed around its perimeter, preferably hermetically (e.g., by an epoxy), to the surrounding wall structure of the housing 119. And this effectively creates an internal cavity within the assembly, formed by the barrier layer itself and the housing. One closed end (e.g., the bottom end) of the cavity provided by the barrier layer and the surrounding wall structure and a top closed end of the cavity provided by the housing. In various embodiments, the electrode and, when present, the interlayer electrolyte, are disposed within that cavity.

With further reference to the innards of the assembly, the barrier layer 206 positively separates the electrode (202a-d) and, when present, the interlayer electrolyte (202a,c,d) from physically contacting the drug reservoir 208. The barrier layer also protects the electrode and the interlayer from coming into contact with various material phases that may be present on the drug side, including the drug reservoir itself and various substances thereof, as well as constituents of the environment about the tissue surface, including ambient air. And the barrier layer also provides protection for the drug reservoir and the tissue by preventing material phases and various substances thereof which are on the electrode side, such as an electrolyte solution of the interlayer and products generated by electrochemical reactions taking place at the electrode, including any byproducts thereof, from reaching the drug reservoir where such material phases or substances can adversely interact with the drug or even come into contact with the tissue, where such contact is generally be undesirable.

The drug reservoir 208 stores the drug that is intended for delivery to the subject and it is a Na⁺ ion conductor. Generally, the drug reservoir comprises a Na+ ion conducting electrolyte solution which provides the conductive medium for Na+ ions to electrically migrate through the reservoir. The drug reservoir is positioned adjacent to the barrier layer second surface and it is generally in contact with that second surface. In accordance with the drug electrode assembly embodiments depicted in FIGS. 2A-2D, the drug reservoir physically contacts and substantially covers at least a portion of the barrier layer second surface. In various alternative embodiments, additional Na+ ion conducting material layers are contemplated between the barrier layer and the drug reservoir, in order to enhance interfacial stability or otherwise improve assembly performance.

The electrode (202a-d) is electrically coupled to the exterior electrical connector 111 and the first surface of the electrode generally faces all three of the barrier layer 206, the drug reservoir 208 and, when present, the interlayer (204a,c,d). In accordance with the instant invention, in each of the three embodiments depicted in FIG. 2A, FIG. 2C and FIG. 2D (all three of which comprise an interlayer) the electrode of the assembly takes a different form.

The electrode 202a depicted in FIG. 2A is a “reactive” sodium electrode of the assist ion. Typically the sodium electrode is a sodium metal electrode, or a sodium metal alloy electrode or a sodium intercalation electrode. Generally it is “aqueous incompatible” and for that matter “chemically incompatible” in contact with water, but the invention is not intended to be limited as such and “aqueous compatible” sodium electrodes are contemplated for use herein.

The electrode 202c depicted in FIG. 2C is a “reactive” electrode that is not of the assist ion. And when electrochemically reduced or oxidized, generally absorbs or desorbs an ion that is not the assist ion (i.e., not Na⁺ ions when the assist ion is Na⁺ ions). Typically, the electrode 202c is a metal electrode, e.g., a Ag, Fe, Cu or Zn metal electrode, or a metal/metal salt electrode, e.g., Ag/AgCl. In a preferred embodiment, the reactive electrode is “aqueous compatible”, and the interlayer electrolyte comprises a Na⁺ ion conducting aqueous electrolyte solution.

The electrode 202d depicted in FIG. 2D is an “inert” electrode, that distributes current through the oxidation and/or reduction of an electroactive chemical species present in an interlayer electrolyte.

With reference to FIG. 2B, the assembly 200b therein does not comprise an interlayer electrolyte, and the electrode 202b while not limited as such is typically a fully solid-state “reactive” electrode of the assist ion and it forms an intimate inorganic solid-state interface in conjunction with the inorganic solid-state conductive medium of the barrier layer. When the assist ion is Na⁺ ions, the electrode 122 is typically a fully solid-state sodium “reactive” electrode, e.g., a sodium metal electrode, a sodium metal alloy electrode, or a sodium intercalation electrode. Because the electrode is a “reactive” electrode of the assist ion, sodium ions (i.e., the assist ions) are absorbed or desorbed by the electrode during drug delivery. In accordance with this embodiment, the sodium electrode takes on the function of assist ion source when the electrode takes the form of a negative electrode, as sodium ions are desorbed directly into the barrier layer, as they move across the intimate solid-state interface. And the electrode takes on the function of assist ion sink when the sodium electrode takes the form of a positive electrode, sodium ions are directly absorbed by the electrode as they migrate out of the barrier layer.

The interlayer electrolyte, when present, (204a,c,d) is interposed between the barrier layer and the electrode, and physically contacts and substantially covers at least a portion (generally a major portion) of the electrode first surface. As depicted in FIGS. 2A, 2C and 2D, the interlayer also physically contacts and substantially covers at least a portion (generally a major portion) of the barrier layer first surface. Generally, the interlayer positively separates the barrier layer from physically contacting the electrode, and the interlayer is itself chemically compatible in contact with both the electrode and the barrier layer. The interlayer electrolyte is sufficiently ionically conductive to support the electrical current which flows through the assembly during drug delivery. In alternative embodiments, additional ion conducting material layers, generally Na+ ion (i.e., assist ion) conducting, may be incorporated between the barrier layer and the interlayer-electrolyte.

With reference to FIG. 2A, since the electrode 202a is “aqueous incompatible”, the interlayer 204a is a non-aqueous Na+ ion conductor. The interlayer 204a can be a solid-state inorganic Na+ ion conductor, e.g., a Na+ ion conducting glass. Alternatively, the interlayer 204a comprises a Na+ ion conducting non-aqueous electrolyte solution, typically a liquid organic electrolyte solution impregnated in a semi-permeable membrane, or a gel electrolyte of which the liquid phase of the gel is the liquid organic electrolyte solution of the interlayer. In this embodiment, both the electrode and the interlayer electrolyte may take on the function of assist ion source or sink during drug delivery. Because the electrode 202a is a sodium electrode that absorbs or desorbs sodium ions during drug delivery, in this embodiment the electrode typically provides the source of assist ions when the electrode takes the form of a negative electrode, and it provides the sink for assist ions when the electrode takes the form of a positive electrode.

With reference to FIG. 2C, because the “reactive” electrode is “aqueous compatible” the interlayer generally comprises a Na+ ion conducting aqueous solution, albeit non-aqueous solutions are contemplated herein.

Lastly, with respect to the drug electrode assembly depicted in FIG. 2D, the electrode 202d therein is “inert” so the interlayer comprises an electrolyte solution that comprises a chemical species that is capable of accepting or donating an electron at the potential applied of the electrode. Generally the electrolyte solution of this interlayer is an aqueous solution, albeit non-aqueous solutions are also contemplated.

With reference to FIGS. 2C and 2D, because the electrode 202c-d is not of the assist ion, sodium ions (i.e., the assist ions) are neither absorbed nor desorbed by the electrode during drug delivery. In accordance with these embodiments, it is the interlayer electrolyte that takes on the function of assist ion source and assist ion sink when, during drug delivery, the electrode takes the form of a negative electrode and a positive electrode, respectively.

Barrier Layer

With general reference to FIGS. 2A-2D, and in accordance with the instant invention, the barrier layer 206 prevents transmission of material phases and various substances thereof from moving across it, while concomitantly allowing Na⁺ ions to electrically migrate into, out of, and through it via a continuous solid-state Na⁺ ion conductive medium. Accordingly, the barrier layer has both barrier and conductive properties.

The barrier layer is generally impermeable to material phases that are adjacent to it and which contact its first and second surface during operation, storage, and manufacture (of the electrode assembly). This includes solids and liquids, and combinations thereof (e.g., semi-solid gels), and preferably fluids (i.e., phases having a liquid or gaseous state of matter). Accordingly, the barrier layer contains no through pores, or passageways whatsoever that would allow the material phase to seep through or otherwise move across the barrier layer.

In accordance with the instant invention, a liquid impermeable barrier layer is at least impermeable to liquid and solid phases, and combinations thereof (e.g., semi-solid gels), preventing any such material phase, which contact its surfaces, from transmitting across it. Preferably, the barrier layer is a fluid impermeable barrier-layer, which is one that is further impermeable to gaseous phases, including ambient air and moisture derived thereof. More preferably, the barrier layer is also impermeable to various substances (e.g., molecules, ions and the like) that are present or make-up the material phase(s) of those substances.

These barrier properties afford a great deal of flexibility in terms of the choice of and the ability to optimize the material phases on either side of the barrier layer. For instance: an electrolyte solution present in the drug reservoir may be aqueous and the electrode may be chemically incompatible in contact with water and, for that matter, with moisture from the ambient air; moreover, the interlayer (when present) may be or comprise a non-aqueous electrolyte solution, and preferably that solution maintains a very low moisture content in order to keep a stable interface in contact with the electrode; moreover, the non-aqueous electrolyte solution of the interlayer may comprise an organic solvent, generally an aprotic liquid organic solvent, the molecules of which are desirably kept from contact with the drug or the tissue surface. Hence, the liquid or fluid impermeable barrier layer is preferably further impermeable to water molecules and/or molecules of organic solvents, including organic liquids, that are present in the electrolyte solution of the interlayer or in that of the drug reservoir. Preferably, the barrier layer is impermeable to all but one substance of the material phases it (the barrier layer) contacts—the only permeable substance being the assist ion, e.g., Na⁺ ions.

In its various forms the barrier layer is preferably also impervious to those material phases and substances thereof, for which it (the barrier layer) contacts and is impermeable (including liquid phases, fluid phases and substances thereof). By impervious it is meant that the referenced phases and substances in contact with the barrier layer on its first and second surfaces are unable to penetrate the solid portion of the barrier layer, for example a water impervious barrier layer is not swelled or infused (e.g., by capillary action) by liquid water, or preferably even water molecules. Accordingly, the liquid or fluid impermeable barrier layer is preferably also impervious to those liquids or fluids for which it is impermeable in contact with. Furthermore, the barrier layer may be both impermeable and impervious in contact with aqueous liquids and water molecules; and/or impermeable and impervious in contact with non-aqueous electrolyte solutions of the drug reservoir and/or those of the interlayer, and preferably the barrier layer is further impermeable and impervious in contact with liquid organic solvents, and even more preferably both impermeable and impervious to the organic molecule of the liquid organic solvent that it contacts.

During device operation, when an electrical current flows into or out of the drug electrode assembly, the barrier layer 206 provides a continuous solid-state conductive medium for Na⁺ ions (assist ions) to electrically migrate through. By solid-state conductive medium it is meant that the medium does not require a liquid phase or for that matter a gel phase to facilitate or bring about assist ion conduction or transport through it (the barrier layer). This can be held in stark contrast with gel electrolytes, which are effectively a continuous liquid phase electrolyte retained within a solid phase, the liquid phase generally providing the medium of ion conduction or at least facilitates that conduction; or in contrast with polyelectrolytes, ionomers, charge selective membranes or ion exchange resins, or the like, which also generally rely on the presence of a continuous liquid phase to facilitate ion conduction. Notably, the presence of a continuous liquid phase, renders gels, polyelectrolytes, ionomers, charge selective membranes and the like, permeable to liquids generally, and inherently permeable to those liquids which make-up the continuous liquid phase. For instance the liquid electrolyte phase of a gel electrolyte, e.g., a hydrogel electrolyte is inherently permeable to water molecules.

Furthermore, in accordance with the instant invention, the barrier layer is solid-state, which includes in its meaning that the barrier layer is not a semi-sold gel. Furthermore, the barrier layer is preferably substantially devoid of liquids, such as liquid water, and the barrier layer may be “dry”, which means that the barrier layer has no water molecules inside it, or if any water molecules are present within the barrier layer, they are present as an impurity, being undesirable and generally inconsequential to the performance and operation of the barrier layer.

During drug delivery, the ionic current flowing through the barrier layer is carried by the electrical migration of Na⁻ ions (the assist ion) so the Na⁺ ion conductivity of the barrier layer needs to be high enough to support the electrotransport current. Generally the Na⁺ ion conductivity of the barrier layer is at least as high as 10⁻⁷ S/cm, more preferably at least as high as 10⁻⁶S/cm, and even more preferably at least 10⁻⁵ S/cm, 5×10⁻⁵ S/cm or 10⁻⁴ S/cm or higher.

In addition to providing a solid-state conductive medium for assist ion (e.g., Na⁺ ion) electrical migration, the ionic conductivity of the barrier layer is species selective (e.g., showing at least one, preferably at least two-, more preferably at least three- or four- or more orders of magnitude greater conductivity for Na⁺ ions than for any other ion). Species selective conductivity discriminates well beyond that of mere charge or size selectivity alone or in combination, because species selectivity encompasses that which is based on the chemical and electronic make-up of the assist ion in combination with the chemical and atomic structure of the solid-state conductive portion of the barrier layer.

The barrier layer is preferably a single-ion conductor of the assist ion. Single-ion conducting barrier layers in accordance with the instant invention have an assist ion transference number of at least 0.95, or at least 0.99, or even at least 0.999. The transference number being defined as the ratio of the assist ion conductivity divided by the total conductivity of the layer, where the total conductivity includes the electronic conductivity plus the ionic conductivity of all ions of the layer. Mixed electronic/ionic conductors are also contemplated.

In order to achieve the requisite properties and functionality of a barrier layer as described above, the barrier layer comprises at least one impervious solid-state electrolyte material which conducts Na⁺ ions (the assist ion). The impervious solid-state electrolyte material is impervious to those material phases and substances thereof for which the barrier layer is impermeable. Furthermore, the impervious solid-state electrolyte material of the barrier layer is inorganic—organic solid-state ion conductors cannot impart the requisite impervious barrier properties required of a solid-state electrolyte material of a barrier layer in accordance with the principles described above because they generally swell with liquid solvents, and conducting ions which are present in an adjacent liquid will generally drag solvated molecules of the liquid along with it as it moves into an organic ion conductor.

The impervious solid-state electrolyte material of the barrier layer is also a species selective ion conductor (e.g., showing at least one, preferably at least two-, more preferably at least three- or four- or more orders of magnitude greater conductivity for the assist ion than for any other ion). Preferably, the impervious solid-state electrolyte material of the barrier layer is a single-ion conductor of the assist ion (single ion conductor as defined for a barrier layer is pertinent here as well). Mixed electronic/assist ion conductors are also contemplated for the impervious solid-state electrolyte material.

The assist ion (e.g., Na⁺ ion) conductivity of the impervious solid-state electrolyte material of the barrier layer is generally at least as high as 10⁻⁶ S/cm, more preferably at least as high as 5×10⁻⁶ S/cm, and even more preferably at least 10⁻⁵ S/cm, 5×10⁻⁵ S/cm or 10⁻⁴ S/cm or higher.

The impervious solid-state electrolyte material of the barrier layer may be an amorphous solid-state inorganic material, such as an inorganic glass, an inorganic crystalline material such as an inorganic polycrystalline ceramic or an inorganic glass-ceramic, having the properties which are consistent with the principles of an impervious solid-state electrolyte material as described above. Accordingly, the inorganic solid-state electrolyte material may be a glass, ceramic, glass-ceramic or combination thereof.

Suitable inorganic solid⁻state Na⁺ ion conducting ceramics include, but are not limited to Nasicon (sodium super ion conductor) materials generally, including Na₁₊ₓZrₓP₃₋ₓSiₓO₁₂ where O≦X≦3, Na₃M₂(PO₄)₃ where M=Sc, Cr or Fe, Na₅RESi₄O₁₂ where RE is Yttrium or any rare earth metal, Na₅SmSi₄O₁₂, Na₅DySi₄O₁₂, Na₃Zr₂Si₂PO₁₂, Na₅GdSi₄O₁₂, NaxTi₃P₆Si₂O₂₅ where O≦X≦2 and beta alumina materials generally including B—Al₂O₃, B″—Al₂O₃, Na₂O0.5Al₂O₃, Na₂O.11Al₂O₃ Na₂O.xAl₂O₃ where 8≦X≦11.

Suitable inorganic solid-state Na+ ion conducting glasses include, but are not limited to, sodium silicates, sodium phosphates (e.g., sodium metaphosphate), sodium sulfides, sodium germinates, sodium borosilicate, Nasiglass (e.g., 33.3Na₂O-16.6ZrO₂-50SiO₂), sodium aluminosilicate, sodium phosphorous-sulfides, sodium borates and combinations thereof. Specific examples include Na₂O—SiO₂, Na₂O—SiO₂—B₂O₃, Na₂O—ZrO₂—SiO₂—P₂O₅, Nasiglass, Na₂S—GeS₂, Na₂S—B₂S₃, Na₂O—B₂O₃—Al₂O₃.

The barrier layer is composed, in whole or in part, of the impervious solid-state electrolyte material or a combination of such impervious solid-state electrolyte materials, suitable examples of which have just been described above. For instance, the barrier layer can be a continuous, monolithic layer of just the impervious solid-state electrolyte material (e.g., as a sintered sheet or glass-ceramic plate). The barrier layer may also comprise additional materials to enhance performance or bring about the requisite properties of a barrier layer consistent with the principles described above.

The solid-state electrolyte material(s) may be distributed uniformly or discretely throughout the barrier layer. For instance, the barrier layer may simply be a compositionally homogenous layer of the impervious solid-state electrolyte material(s). In other instances the barrier layer may be a multi-layer composite laminate of two or more layers, each layer comprising an impervious solid-state electrolyte material. The first layer of the composite laminate forming the barrier layer first surface and optimized for its chemical compatibility in contact with constituents on the electrode side of the assembly, and the second layer forming the barrier layer second surface and optimized for its chemical compatibility in contact with constituents on the drug side. For instance, the barrier layer may be a composite laminate of a first beta-alumina layer and a second Nasicon layer, e.g., the Nasicon layer facing the drug side of the assembly and the beta alumina layer facing the electrode side. Or the barrier layer may be a laminate composite of a sodium ion conducting ceramic layer and a sodium ion conducting glass layer. For instance the barrier layer may be a laminate composite of a relatively thick ceramic layer, e.g., beta alumina, coated with a relatively thin sodium ion conducting glass layer. And the thin conducting glass layer may be coated onto one or both sides of the ceramic layer. Without limitation, the relatively thick ceramic layer is generally in the range from about 10 microns to 1000 microns, more typically in the range of about 25 to 500 microns, and even more typically in the range of about 50 to 250 microns; and the relatively thin glass layer is generally in the range from about 0.25 to 5 microns, more typically in the range of about 0.5 to 2.5 microns and even more typically about 1 micron thick.

Furthermore, even though the barrier layer is impermeable to liquid phases and preferably to fluid phases, it is possible, and generally the case, that the barrier layer contains some solid pores as well as defects—just so long as those pores or imperfections do not provide passage for liquids, and preferably fluids, to move through and across the layer. In various embodiments the barrier layer is dense having solid porosity less 20%, more preferably less than 10% and even more preferably less than 5%.

The barrier layer may comprise additional material components which may or may not be conductive to the assist ion. For instance, various materials can be incorporated into a barrier layer to enhance or render barrier properties to the layer, generally to improve mechanical properties, or facilitate processing. For instance, processing aids, such as ceramics (e.g., Na₂O) or glasses (e.g., silicates) can be incorporated to improve densification upon sintering the layer; and inert polymers (e.g., polyethylene, polypropylene) can be distributed within the layer to improve mechanical integrity. The barrier layer may further comprise a filler component material (e.g., an epoxy resin or glass) used to close off any through porosity.

The barrier layer may be fabricated as a freestanding layer consistent with the principles, compositions and structures described above for a barrier layer. In accordance with the instant invention freestanding barrier layers can be fabricated by any technique known for fabrication of inorganic glasses, ceramics, and glass-ceramics in the form of a layer (e.g., a sheet, plate, membrane, etc.), including but limited to quenching a melt of the impervious solid-state electrolyte material to form a glass, sintering (e.g., tape casting followed by sintering) of ceramic or glass-ceramic powders of the solid-state electrolyte material, and glass-ceramic processing of the solid-state electrolyte material, which generally entails the steps of melting and quenching to form a glass, followed by annealing and a crystallization heat treatment.

Freestanding layers and methods of making such freestanding layers consistent with the principles described above for a barrier layer and which can be usefully employed as a Na+ ion conducting barrier-layer in a drug electrode assembly in accordance with the instant invention are disclosed in the following US Patents and Patent Applications, all of which are hereby incorporated by reference herein: U.S. Pat. No. 7,255,961 to Schucker; U.S. Pat. No. 3,404,036 to Kummer; U.S. Pat. No. 3,896,019 to Mitoff; U.S. Pat. No. 3,976,554 to Powers; U.S. Pat. No. 5,290,405 to Joshi; U.S. Pat. No. 5,580,430 to Balagopal; U.S. Pat. No. 3,901,733 to Toy.

Residual through porosity and/or the like, which may be present in a freestanding barrier layer, including any of the barrier layers incorporated by reference above, can be closed off by incorporating into any such through-pores a filler component (e.g., an epoxy resin), which effectively plugs-up the holes, rendering the layer impermeable and preferably impervious. Methods for closing off residual through porosity of a barrier layer, and associated filler compositions are described in applicant's commonly assigned U.S. Patent Application Pub. No. US 2007/0172739 to Visco, and is hereby incorporated by reference herein for all that it discloses.

The Drug Reservoir

With reference to FIGS. 2A-2D, the drug reservoir 208 comprises a drug that is intended for delivery to the subject and it conducts assist ions. The assist ion conducting drug reservoir may take a variety of forms, including any structure capable of retaining the drug and an assist ion conducting electrolyte solution, and which allows the drug to permeate under an electrical field or electrical current, so that it (the drug) can reach the tissue surface, and it is sufficiently ionically conductive to support the electrical current that flows through the assembly during drug delivery. Generally, it is the drug reservoir on the drug side of the barrier layer that takes on the function of assist ion source and assist ion sink when the electrode takes the form of a positive and a negative electrode, respectively.

The drug reservoir is positioned adjacent to the barrier layer second surface and it is generally in contact with that second surface. In accordance with the drug electrode assembly embodiments depicted in FIGS. 2A-2D, the drug reservoir physically contacts and substantially covers at least a portion of the barrier layer second surface. In various alternative embodiments, additional assist ion conducting material layers are contemplated between the barrier layer and the drug reservoir, in order to enhance interfacial stability or otherwise improve assembly performance.

The drug reservoir comprises an assist ion conducting electrolyte solution and a drug. The assist ion conducting electrolyte solution is distributed throughout the reservoir and can be formulated, without limitation, by dissolving at least one assist-ion salt in at least one solvent or by solvating at least one assist-ion containing polyelectrolyte (e.g., an ionomer, or ion exchange resin, or the like) with at least one solvent, or a combination thereof. The assist ion exists in the electrolyte solution in the form of an ion that is mobile in the solution, and therefore is capable of electrically migrating throughout the reservoir. The drug can be loaded into the reservoir, without limitation, as a drug solution or drug suspension or drug emulsion or drug layer. The drug may be distributed fairly uniformly throughout the reservoir, for instance the drug may dissolved, solvated, suspended or otherwise dispersed in the assist ion conducting electrolyte solution, or the drug may be incorporated in the reservoir in the form of a drug layer, e.g., the drug blended or dispersed or otherwise distributed in a polymeric layer which may be swelled or gelled in contact with the assist ion conducting electrolyte solution, or a combination thereof. When the drug is ionic, it exists in the electrolyte solution in the form of ions, and can be introduced into the solution as a salt, base, or acid.

Without limitation, the drug reservoir can take the configuration of a structural cavity or receptacle that contains the drug and the assist ion conducting electrolyte solution, or it (the reservoir) can take the form of a pad such as a gauze or felt or reticulated foam containing the drug and soaked with an assist ion conducting electrolyte solution, or the drug reservoir may further comprise a matrix material that retains the drug and the electrolyte solution, e.g., a drug containing gel electrolyte.

The matrix material of the drug reservoir, when present, can be organic, inorganic and naturally occurring or synthetic material or combination of such materials that gels or swells or otherwise is able to retain the electrolyte solution and the drug. The matrix material can be, without limitation, a synthetic polymer including poly(acrylamide), poly(2-hydroxyethyl acrylate), poly(2-hydroxypropyl acrylate), poly(n-vinyl-2-pyrollidone), poly(n-methylol acrylamide), poly(diacetone acrylamide), poly(2-hydroxyethyl methacrylate), poly(2-hydroxy propyl methacrylate), poly(vinyl alcohol), poly(ethylene oxide), poly(propylene oxide), and poly(allyl alcohol); or a naturally occurring polymers including cellulose ethers, methyl cellulose ethers, cellulose and hydroxylated cellulose, methyl cellulose and hydroxylated methyl cellulose, gums such as guar, locust, karaya, xanthan and gelatin and agar.

In various embodiments, the drug reservoir is the drug containing pad or gel electrolyte itself, such as, but not limited to, polymer gels (e.g., a hydrogel) or polymer matrices imbibed with the electrolyte solution and the drug. Preferably the drug reservoir should retain its general shape and inhibit liquid (e.g., water) loss by evaporation. Polymers and gels suitable to retain electrolyte solutions and drugs are well known to those of skill in the art of iontophoretic devices.

Furthermore, the electrolyte solution of the drug reservoir may itself be formulated by adding a solvent, typically a liquid solvent (e.g., water), to a matrix material which contains an electrolyte group, such as a polyelectrolyte (e.g., an ionomer) or ion exchange material, and the matrix material interacting with the solvent (e.g., by plastisizing or dissolution) brings to bear mobile assist-ions in the solution. For instance, the matrix material can be a polyelectrolyte such as an ionomer, e.g., when the assist-ion is sodium ion (Na⁺) the ionomer can be the sodium form of Nafion (a sulfonated tetrafluorethylene copolymer), and the solvent can be water or it can be non-aqueous, e.g., a polar non-aqueous biocompatible liquid solvent.

The solvent(s) of the assist ion conducting electrolyte solution can be essentially any solvent, preferably biocompatible, for which the assist-ion salt can be dissolved to an effective concentration or that is capable of solvating a matrix material to an effective degree to achieve an electrolyte solution having a sufficient assist ion conductivity and concentration of mobile assist ions that is capable of supporting the electrical migration of assist ions during drug delivery, and the solvent should also be chemically compatible in contact with the drug. In various embodiments the solvent of the electrolyte solution is water, and the electrolyte solution is aqueous. There are several reasons for using an aqueous electrolyte solution, including that water is a biocompatible highly polar solvent and therefore preferred for ionizing or solubilizing many salts, including drug salts. However, the invention should not be limited as such and the electrolyte solution of the drug reservoir may be a non-aqueous electrolyte solution, where the assist ion salt is dissolved in an appropriate organic solvent (s), for instance liquid organic solvents including polyethylene glycols and the like. Non-aqueous electrolyte solutions can be particularly useful when the drug may have chemical stability problems in contact with water or such problems arise after extended storage, or a non-aqueous electrolyte solution can be useful to improve interfacial stability between the drug reservoir and the barrier layer. Moreover, the electrolyte solution may comprise a mixture of solvents including water and at least one or more non-aqueous solvents. Suitable non-aqueous solvent(s) are generally biocompatible in contact with the target tissue, and these include, without limitation, protic and aprotic solvents such as ethers (e.g., polyethylene glycol and diphenyl ether); alcohols (e.g., decanol, dodecanol and oleyl alcohol); carboxylic acids (e.g., oleic acid); esters (e.g., ethyl decanoate, dibutylphthalate and the like). Moreover, suitable non-aqueous solvents, while generally liquids, should not be limited as such, and can be solids, e.g., an assist-ion conducting polymer for which an assist-ion salt can be dissolved such that the assist-ions, once dissolved, are sufficiently mobile within the polymer framework, e.g., when the assist-ion is sodium ion (Na⁺), a Na⁺ ion conducting polyethyelene oxide polymer can be the electrolyte solution, generally swelled with a liquid solvent to enhance its conductivity.

The electrolyte solution can be formulated by dissolving an assist ion salt(s) in the solvent(s) of the electrolyte solution to an effective concentration that is sufficient to support assist ion conduction in the reservoir during drug delivery and support the electrical migration of assist ions across the barrier layer. Salts suitable for use as an assist-ion salt include, but are not limited to, ionic compounds of the assist-ion, preferably of excellent biocompatibility, and this includes drug salts of the assist-ion, which are salts which contain both the drug ion and the assist ion, generally one being the cation and the other the anion of the salt, and polysalts of the assist-ion. For instance, when the assist-ion is a simple metal cation, suitable assist cation salts include drug salts (DM) where D is the drug anion and M is the assist cation (e.g., Na⁺ ion); and polysalts (Poly-M) where Poly is the charged polymer backbone and M is the assist ion; as well as chlorides (M-Cl); acetates (M-CH₃COO); carbonates (M₂-CO₃); citrates M-H₂(C₃H₅O(COO)₃); hydroxides (M-OH); nitrates (M-NO₃); phosphates (M₃-PO₄); and sulfates (M₂-SO₄), where M is the simple metal assist cation, e.g., M is Na, Li, K, Ag, and Cu, for example Na.

Dissolution of the assist ion salt into the solvent(s) brings to bear not only mobile assist ions into the electrolyte solution but counter ions of opposite charge polarity as well. Depending on the type of drug intended for delivery, particularly e.g., ionic drugs, and on the method of delivering the drug ions, particularly when the assist-ions and the drug ions are of opposite charge polarity and electrically migrate in opposite directions during drug delivery, the presence of extraneous counter-ions (i.e., counter-ions that are not assist ions or drug ions) that are mobile in the reservoir is generally undesirable, and this is because mobile extraneous counter ions can compete with drug ions for delivery to the target tissue, and this competition can markedly lower drug delivery efficiency.

For the delivery of ionic drugs, and particularly for the delivery of ionic drugs when the assist ion moves from the drug side to the electrode side during drug delivery, it is preferable to utilize an assist-ion salt that once dissolved in the electrolyte solution yields mobile assist ions and extraneous counter ions having lower mobility than that of the drug ions, or the counter ions are substantially immobile in the electrolyte solution, or the counter ions are reacted in the electrolyte solution to form a substantially immobilized reaction product, or to utilize an assist-ion salt wherein the counter ion is the drug ion itself, and by this expedient extraneous ions are not generated as a result of salt dissolution, and in such an instance, of course, the drug exists in the electrolyte solution as a drug ion that is mobile therethrough.

By one expedient, competitive ion effects can be reduced by formulating the electrolyte solution of the reservoir using a sodium ion polysalt, which when mixed with the solvent of the electrolyte solution interacts with the solvent and beings to bear mobile sodium ions and a negatively charged polymer backbone that, by virtue of its relatively high molecular weight, is substantially immobile in the electrolyte solution, or at least less mobile than the negatively charged drug anions, and the charged polymer can be cross-linked to further reduce its mobility. In like manner, the matrix material of the reservoir can be a sodium polyelectrolye as described above, for instance an ionomer, e.g., when the assist ion is sodium ion (Na⁺) the ionomer can be the sodium form of Nafion. By this expedient, the electrolyte solution is formed by incorporating a solvent, generally a liquid solvent (e.g., water or a non-aqueous biocompatible liquid) into the ionomer which effectively plastisizes the polymer and the presence of the solvent phase about the polymer facilitates sodium ion mobility, the drug can be dispersed or otherwise dissolved in the solvent, and thereby intermixed within the ionomer. As it concerns the terms which define the constituents of the electrolyte solution, the polyelectrolyte or ionomer is considered herein to be the solute of the electrolyte solution, regardless of whether the volume of ionomer in the electrolyte solution is greater than that of the solvent, e.g., water. When the assist-ion is a simple metal cation, suitable polyelectrolytes include poly(sodium styrene sulfonate), as well as those disclosed in U.S. Pat. No. 5,633,098 and which include polysiloxane, polymethacrylate and poly(alkylene oxide) as well as sulfonated tetrafluorethylene copolymer. The degree to which a polyelectrolyte or ionomer dissociates in contact with the solvent can vary, and in accordance with the instant invention any degree which brings to bear mobile assist ions in the electrolyte solution is sufficient.

By another expedient competitive ion effects can be reduced or for that matter mitigated, by formulating the electrolyte solution by dissolving a drug salt of the assist ion, e.g., a sodium drug salt, thereby yielding sodium ions and negatively charged drug ions that are mobile in the electrolyte solution. For instance, a drug salt of the assist ion can be an ionic compound having a cationic drug as the cation of the salt and an assist anion as the anion of the salt, or vice-versa an anionic drug as the anion of the salt and an assist cation as the cation of the salt, e.g., a drug salt of the assist ion comprising an anionic drug and Na+ assist ions.

Additional, supporting electrolyte salts, which may not be an assist ion salt, can be added to the drug reservoir to enhance the ionic conductivity of the electrolyte solution, including tetra-alklylammonium salts such as tetra-butylammonium (TBA) salts including TBA chloride, bromide, iodide or sulfate, as well as tetra-ethylammonium (TEA) salts including TEA hydrogen sulfate or hydrogen carbonate and combinations thereof.

The supporting electrolyte solutions may contain other chemical species which are known to those of skill in the art, to effect various properties of the electrolyte reservoir including surfactants, buffers, osmolarity adjusters (e.g., polyethylene glycols, sugars), antibiotics, penetration enhancers (e.g., alkanols), stabilizers, anti-fungal compounds such as paraben derivatives, enzyme inhibitors, preservatives, thickening agents.

The amount of drug incorporated in the drug reservoir is at least that amount which is effective to bring about the drugs desired effect when delivered to the subject. As noted above the terms “drug,” “drug specie(s)” or “biologically beneficial agent” or more simply “agent” or “agent specie(s) are used interchangeably and are intended to have their broadest interpretation as any agent that elicits a biological response from a subject being treated with the agent, including the delivery of an agent for purposes other than to treat some condition, for example to facilitate diagnosis. The assist ion referred to herein is an ion that facilitates drug delivery and is itself generally not intended nor considered to be the drug or the biologically beneficial agent. However, the invention is not necessarily intended to be limited as such, and in some instances it is possible that the assist ion can also act as a drug. Examples of biologically beneficial agents (i.e., drugs) include therapeutic agents, pharmaceutical agents (e.g., therapeutic compounds, pharmaceutical salts) non-pharmaceuticals (e.g., cosmetic substance and the like), vaccines, immunological agents, local or general anesthetics or painkillers, antigens or proteins or peptides such as insulin, chemotherapy agents, and anti-tumor agents. The term drug encompasses anesthetics, analgesics, immunogens, allergens, opiods and The term “drug” further encompasses prodrugs, metabolites, analogs, and the like. The term drug further encompasses chemicals such as adjuvants which modify the effect of another agent or drug with few if any direct effect when given by itself. For instance an adjuvant may increase the potency or efficacy of a pharmaceutical or affect an immune response.

Drugs useful in the present invention include any pharmaceutical compound or chemical that is capable of being ionized or converted to a charged form, as well as electrically neutral agents, molecules, or compounds capable of being delivered by electrotransport. In general this includes drugs in all of the major therapeutic areas including, but not limited to, those which are disclosed in U.S. Pat. No. 5,871,460 to Phipps et al. and U.S. Patent Pub. No. US 2008/0027369 to Carter et al. Non-limiting examples include, anti-infectives, analgesics, anesthetics, antiarthritics, antiasthmatic agents, antidepresents, antidiabetic agents, antidiarrheals, antihistamines, anti-inflammatory agents, antimotion sickness, antimigraine, antineoplastics, antiparkinsonism.

Non-limiting examples of such agents include lidocaine, articaine, and others of the caine class, morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptaminel receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of agents include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N,N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (NOVOCAINE®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

First Drug Electrode Assembly Embodiment

With specific reference to a preferred embodiment of a first drug electrode assembly embodiment depicted in FIG. 2A, the electrode 202a is a sodium “reactive” electrode that is or comprises a solid-state electroactive component material that absorbs Na⁺ ions when it is electrochemically reduced and desorbs Na⁺ ions when it is electrochemically oxidized. Particularly suitable solid-state sodium electroactive materials include sodium metal, sodium alloys (including both sodium metal and sodium semi-metal alloys) and sodium intercalation compounds. A sodium electroactive intercalation material absorbs Na+ ions when it is electrochemically reduced via an intercalation reaction and desorbs Na+ ions when it electrochemically oxidized via a de-intercalation reaction. Na+ ions absorb into a sodium electroactive alloy when it is electrochemically reduced via an alloying reaction, and Na+ ions are desorbed from a sodium electroactive alloy when it is electrochemically oxidized. And electroactive sodium metal desorbs Na+ ions via a metal striping reaction when the electrode is negative and sodium metal is oxidized and when the sodium metal is reduced, Na+ ions are absorbed by the electrode via a sodium metal plating reaction. Suitable solid-state electroactive sodium metal alloys include, but are not limited to, metal and semi-metal alloys of the sodium metal, such as binary and ternary sodium metal alloys with Ca, Mg, Sn, Ag, Zn, Bi, Al, Cd, Ga, In. Specific examples of preferred sodium metal alloys include sodium aluminum alloys, sodium silicon alloys, sodium tin alloys, sodium silver alloys and sodium lead alloys.

Suitable solid-state electro-active sodium intercalation materials include, but are not limited to, carbons, metal chalcogenides (e.g., oxides and sulfides), metal phosphates, and metal silicates—especially, carbons, transition metal oxides, transition metal phosphates and transition metal silicates. Particularly suitable solid-state sodium intercalation compounds include cobalt oxides, manganese oxides, nickel oxides, vanadium oxides, titanium oxides, iron oxides, tungsten oxide, iron phosphates, transition metal ion doped beta“-beta-alumina compounds, and carbon based intercalation materials such as petroleum coke and graphite.

Particularly suitable solid-state sodium electroactive intercalation materials include CoO₂, V₂O₅, V₆O₁₃, WO₃, NaNiO₂, NaFePO₄, NaFe₃(PO₄)₃, NaNiₓCo₁₋ₓO₂ and NaMn₂O₄ NaₓC, NaₓCoO₂, Na_{1.68}Li_{0.32}Al_{10.41-y}Fe_{y}O₁₈, NaₓC₆ with 6<=x<=0, Na_{1.66}Mg_{0.67}Al_{10.33-y}Fe_{y}O₁₇. Specifically cobalt oxides such as CoO₂, sodium cobalt bronzes such as NaₓCoO₂ such as Na_{0.7}CoO₂, manganese oxides and manganese oxide bronzes such as Na_{0.44}MnO_{2,} Na_{0.44}Z_{y}Mn_{1-y}O₂ wherein Z is a metal capable of substituting for manganese in the orthorhombic structure such as titanium, zirconium, hafnium, vanadium, niobium, tantalum, and y is 0 to 60 atomic %. (see, e.g., U.S. Pat. Nos. 5,916,710; 5,558,961; and 5,443,601 which are incorporated herein by reference for all purposes).

The sodium reactive electrode can be a layer of sodium metal foil, or a coating of sodium electroactive material on a suitable current collector. For instance the electroactive sodium (sodium metal, sodium metal alloy, or sodium intercalation material) may be coated onto a suitable metal foil (e.g., Cu, Al, Stainless Steel, or the like) to the desired thickness sufficient to supply enough capacity to deliver an effective amount of medicament to the subject.

The sodium electrode 202a may further comprise other materials, which are incorporated to improve or enhance electrode performance, for example, electronically conductive and ionically conductive additives, or a binder may be added to enhance mechanical properties such as the overall structural integrity of the electrode.

Sodium intercalation electrodes can be a component mixture of an electroactive component (e.g., sodium intercalation material) intermixed with an electronically conductive component (e.g., conductive carbons such as acetylene black, graphite and the like) and a binder component, generally a polymer (e.g., a PVdF, PEO, PTFE or the like) which provides the mechanical integrity to the coated electrode. The electrode may further comprise polymeric materials (co-polymers of PVdF-HFP PEO), which swell or gel in contact with a liquid electrolyte solution. The electrode can be formed by roll coating a slurry mixture of these components suspended or otherwise dispersed in a carrier liquid onto a suitable current collector such as a copper, aluminum, or stainless steel foil. The electrode can be, and generally is, in the form of a layer having a first surface facing the barrier layer and a second surface facing and generally adhered to the electronic current collector which is electrically coupled to exterior connector 111. Composite intercalation electrodes as described above are generally somewhat porous, and the ionic conductivity of the coating can be enhanced when the pores are filled with a suitable non-aqueous ion conducting liquid electrolyte, typically the electrolyte solution of the interlayer. For instance, when the assembly is fabricated, the liquid interlayer electrolyte solution can be applied to the first surface of the electrode in sufficient quantity to soak or at least wet the electrode first surface and the internal pore surfaces and fill the pores, the barrier layer may then be placed directly onto the electrolyte wetted surface of the electrode. Or, more typically, the interlayer electrolyte further comprises a material layer that retains the electrolyte solution such as a semipermeable membrane layer (e.g., a microporous separator layer) or gel electrolyte layer, and during fabrication this layer can be placed directly onto the wetted electrode. An additional amount of electrolyte solution may be added on top of the separator or gel layer once it has been placed on the electrode first surface, and this to enhance wetting of the interlayer.

The sodium “reactive” electrode and for that matter its electro-active material (e.g., electro-active sodium) may, at least initially, not contain sodium until the device actively starts delivering drug under an electrotransport current. For instance, the electrode could be an electronically conducting substrate suitable for the plating of sodium metal (e.g., sodium), an a sodium alloying metal (e.g., Sn) or a sodium intercalation material (e.g., Carbon or V2O5) all initially free of sodium until drug delivery is initiated and electro-reduction leads to Na+ ions absorbing via alloying or intercalating into the electoactive material, or plating as a result of the electrode reduction.

With continued reference to FIG. 2A, the interlayer 204a is a non-aqueous Na+ ion conductor. The non-aqueous interlayer may comprise a non-aqueous electrolyte solution of a sodium salt dissolved in a non-aqueous solvent to an effective amount that the Na⁺ ion conductivity of the interlayer is sufficient to support the electrotransport current through the assembly. The solvent is generally organic, for instance an organic polymer or organic liquid. Generally, it is an organic liquid (typically an aprotic organic liquid), and the electrolyte solution of the interlayer is a Na₊ ion conducting liquid organic electrolyte solution. The electrolyte solution of the interlayer is chemically compatible in contact with the electrode, and chemically compatible in contact with the first surface of the barrier layer.

Aprotic liquid organic solvents suitable for use in the interlayer 204a include, but are not limited to, organic carbonates, ethers, lactones, sulfones, etc., and combinations thereof, such as EC, PC, DEC, DMC, EMC, 1,2-DME or higher glymes, THF, 2MeTHF, sulfolane, and combinations thereof.

Suitable sodium salts include, but are not limited to, NaPF₆, NaBF₄, NaAsF₆, NaClO₄, NaSO₃CF₃ or NaN(SO₂C₂F₅)₂. The salt concentration of the electrolyte solution is commonly selected based on optimizing the sodium ion conductivity; generally, the concentration is in the range of about 0.2 molar to 1.5 molar, most commonly about 1 molar.

The electrolyte solution of the interlayer has a Na⁺ ion conductivity that is sufficient to support the electrotransport current through the device, preferably it is at least as high as 10⁻⁷ S/cm, more preferably at least as high as 10⁻⁶ S/cm, and even more preferably at least 10⁻⁵ S/cm, 5×10⁻⁵ S/cm or 10⁻⁴ S/cm or higher. The non-aqueous electrolyte solution may be dry, and when the electrode is “aqueous incompatible” or for that matter chemically incompatible in contact with the water, the non-aqueous electrolyte preferably has a water content that is less than 500 parts per million (ppm), more preferably less than 250 ppm, and even more preferably less than 100 ppm.

Generally, the interlayer positively separates the electrode from physical contact with the barrier layer, and the interlayer is chemically compatible in contact with both the barrier layer first surface and the electrode first surface. The positive separation may be derived by the formation of cavity formed between the electrode and the barrier layer and the assembly walls, more commonly the interlayer comprises a material separator chemically compatible in contact with both the barrier layer and the electrode or the interlayer itself may take the form of a gel electrolyte.

The interlayer may comprise or take the form of a semi-solid gel electrolyte containing the non-aqueous liquid electrolyte solution imbibed in a solid, generally polymeric, phase (e.g., a solid matrix structure). Suitable solid phase gel constituents include, but not limited to, polyvinylidine fluoride (PVdF) compounds, hexafluropropylene-vinylidene fluoride copolymers (PVdf-HFP), polyacrylonitrile compounds, cross-linked polyether compounds, polyalkylene oxide compounds, polyethylene oxide compounds, and combinations and the like may be added to gel the interlayer electrolyte solution.

The interlayer may comprise, a material separator layer, such as a semipermeable membrane (e.g., a microporous membrane) impregnated with the electrolyte solution of the interlayer or for that matter with the gel electrolyte, as described above. The material separator layer can also be a polymer layer that is swelled by the interlayer electrolyte solution, e.g., the organic liquid interlayer electrolyte solution. Typically, interlayer is a microporous polymer (e.g., a polyolefin microporous layer) the pores of which contain and are wetted by a non-aqueous Na₊ ion conducting aprotic liquid organic electrolyte solution. Suitable semi-permable separator layers include micro-porous polymers such as micro-porous polypropylene and/or micro-porous polyethylene, such as a Celgard micro-porous separator.

Second Drug Electrode Assembly Embodiment

In accordance with a second drug electrode assembly 200b embodiment as depicted in FIG. 2B, the electrode 202b can be a fully solid-state electrode of a sodium electroactive material, for instance sodium metal, sodium alloy (including metals and semi-metals) or electroactive sodium intercalation material, e.g., a sodium metal alloy. In accordance with this embodiment, the fully solid-state sodium electrode in physical contact with the barrier layer forms an intimate inorganic solid-state interface between itself and the solid-state inorganic conductive medium of the barrier layer. In accordance with the instant embodiment, electroactive sodium suitable for use as the solid-state sodium electrode described herein includes the electroactive sodium material described above and with reference to the sodium electrode depicted in FIG. 2A, and as such will not be repeated here.

Without limitation, one method of forming an intimate solid-state interface between the barrier layer and the sodium electrode 202b is by depositing the electroactive sodium material directly onto the barrier layer first surface by physical or chemical vapor deposition (PVD or CVD), including thermal evaporation of sodium metal or a sodium metal alloy, or sputtering of a sodium intercalation material, the interface may also be formed by melt casting sodium metal or the sodium metal alloy directly onto the barrier layer.

Third Drug Electrode Assembly Embodiment

In accordance with a third drug electrode assembly 200c embodiment as depicted in FIG. 2C, the electrode 202c is an “aqueous compatible” “reactive” electrode that when electrochemically reduced or oxidized, generally absorbs or desorbs an ion that is not the assist ion of the assembly (i.e., not Na+ ions when the assist ion is Na+ ions). Typically such a reactive electrode generally comprises a metallic salt in contact with a metal. For example silver chloride in contact with metallic silver (Ag/AgCl electrode) or iron chloride in contact with metallic iron (Fe/FeCl electrode). During device operation, the Ag/AgCl electrode may be electrochemically reduced or oxidized, as AgCl on the surface of the metallic silver electrode is reduced to give silver metal and chloride anion or when the electrode is oxidized silver goes to silver ions as follows:

AgCl+e⁻ [symbol] Ag+Cl⁻

Ag−e⁻ [symbol] Ag⁺

Electrodes suitable for use in the drug assembly depicted in FIG. 2C include, but are not limited to, those aqueous compatible electrodes that undergo the following electrochemical reactions: metal plating/striping reactions and displacement reactions, which generally involve reduction and oxidation of a metal/metal salt electroactive component. Particularly suitable metal electrodes include the following metals (e.g., Ag, Cu, Fe, and Zn) which are plated from the interlayer electrolyte solution onto the electrode when the electrode is reduced and are striped off of the electrode into the interlayer electrolyte solution when the electrode is oxidized electrochemically. Particularly suitable electroactive metal/metal salt electrodes comprise Ag/AgCl, Fe/FeCl, Cu/CuCl and the like. These electroactive metal/metal salt compounds generally desorb anions of the salt (e.g., Cl⁻ ions) when they are electrochemically reduced and desorb metal cations of the salt (e.g., Ag⁺) when they are electrochemically oxidized, generally, in the electrochemical process a metal salt precipitate is formed (e.g., AgCl).

When the electrode 202c is “aqueous compatible”, the interlayer electrolyte 204c is generally an aqueous solution that contains the charge-compensating ion that the electrode absorbs or desorbs in order to maintain electrical charge neutrality. In the case of a Ag/AgCl electrode, chlorine ions (Cl−) are desorbed into the electrolyte solution of the interlayer when the electrode takes the form of a positive electrode, and silver ions are desorbed when the electrode is oxidized, albeit if the electrolyte solution contains chlorine ions, the Ag+ ions are almost immediately precipitated as AgCl at the electrode.

In the instant embodiment, the assist ion is sodium ions and they are neither absorbed nor desorbed by a Ag/AgCl electrode during drug delivery, so it is the interlayer electrolyte which functions as the sodium ion source and as the sodium ion sink depending on the polarity of the electrode during drug delivery. When the interlayer electrolyte takes on the function of the source, it contains a sufficient amount of mobile sodium ions to support the electrical migration across the barrier layer, and when it functions as the sink sodium ions which enter the interlayer from the barrier layer may and generally do exist in the electrolyte as ions, but the invention is not limited as such, and it is contemplated that the sodium ions may precipitate out of interlayer electrolyte solution if their concentration gets too large over the span of drug delivery. Generally, the electrolyte solution of the interlayer is an aqueous salt solution of NaCl, or when precipitation of AgCl is undesirable the aqueous salt solution of the interlayer may comprise a salt which has a sufficiently high solubility constant to prevent or lessen the precipitation of silver salts when the electrode takes the form of a negative electrode, for instance the interlayer electrolyte solution may be a sodium nitrate solution. And when the electrode takes the form of a positive electrode, chlorine ions are desorbed in an electro-reduction reaction, and in this instance a NaCl solution is particularly neat and simple since Cl− ions enter the solution from the electrode and Na ions enter the solution as they migrate across the barrier layer from the drug side.

Furthermore, iontophoretic devices that employ Ag/AgCl electrodes for anionic drug delivery generally include in the electrolyte solution adjacent to the electrode a substance which immobilizes the desorbed Cl⁻ ions, for instance Ag+ ions in solution, in order to minimize competitive ion effects. Indeed, due to the very low solubility constant of AgCl, the presence of Ag+ ions in solution leads to the almost immediate precipitation of AgCl adjacent the electrode once the Cl⁻ is desorbed. If not for this precipitation, the Cl⁻ ions generated at the electrode when it (the electrode) is reduced would compete with the anionic drug for delivery to the subject. Unfortunately, precipitation reactions can lead to clogging of the electrode pore structure and undesirable electrode volume changes, which generally leads to decreased electrode capacity. In accordance with the instant invention, because the barrier layer 206 prevents the permeation of Cl⁻ ions across it, competition effects can be mitigated without using chemical methods such as precipitation and the like. And this leads to various advantages not the least of which is increased electrode capacity which translates into longer operational lifetime of the assembly and the device for which it is integrated into, e.g., the electrotransport device. With continued reference to the third embodiment depicted in FIG. 2C, the interlayer 204c comprises an electrolyte solution that is generally aqueous, but is not limited as such and non-aqueous electrolyte solutions are contemplated. The electrolyte solution physically contacts and substantially covers at least a portion of the electrode surface in order to facilitate the electrochemistry at the electrode, and the absorption or desorbing of the ion (e.g., Cl⁻ ions) by the electrode, e.g., Ag/AgCl electrode.

Fourth Drug Electrode Assembly Embodiment

In accordance with the fourth embodiment of a drug electrode assembly 140 of the instant invention as depicted in FIG. 2D, the electrode 202d is not a reactive electrode, rather it is an “inert” electrode which facilitates electron transfer to an electroactive chemical species present in the interlayer electrolyte 202d, the chemical species being capable of accepting or donating an electron at the potential applied between (or generated by) the first and second electrode assemblies.

Suitable inert electrodes include, carbon based inert electrodes, as well as others including, stainless steel, gold, platinum, capacitive carbon or graphite.

The interlayer 204d comprises an electrolyte solution which comprises the electroactive chemical species (i.e., redox species) capable of accepting or donating an electron at the potential of the electrode. The electrolyte solution of the interlayer is generally aqueous, but it is not so limited and non-aqueous electrolyte solutions are also contemplated. When an inert electrode is employed and the interlayer electrolyte is aqueous, water can be the redox species which is electrochemically oxidized or reduced. Or, more generally, the interlayer (aqueous or otherwise) comprises a redox specie(s) that is oxidized at the applied electrode potential such as an anti-oxidant, e.g., ascorbic acid (vitamin C) and tocopherol (vitamin E), or a redox specie(s) that is reduced at the applied electrode potential, e.g., ferrocenium salts dissolved in the electrolyte solution. In some embodiments, the redox species has a lower potential than that of, for example, water or the electrolyte solvent, so that the redox species is reacted rather than having, for example, hydrolysis of water taking place at the electrode. Furthermore, because the interlayer and its constituents are prevented from moving into the drug reservoir, electroactive chemical species which may be incompatible in contact with the drug, or undesirable in contact with the tissue surface, are contemplated for use herein.

Again, because the electrode does not absorb or desorb assist ions during drug delivery, the interlayer electrolyte 204d, and in particular its electrolyte solution, takes on the function of assist ion source and assist sink during drug delivery. Generally the aqueous solution of the interlayer comprises a NaCl salt solution of sufficient sodium ion conductivity and concentration to support the electrical current through the assembly during drug delivery.

With reference to FIGS. 2A-2D, in certain particular embodiments the drug electrode assembly 200a-d depicted therein is configured to deliver an anionic drug species when the electrode 202a-d takes the form of a positive electrode during drug delivery. And in other particular embodiments the drug electrode assembly 200a-d is configured to deliver a drug species, e.g., a neutral drug species, when the electrode 202a-d takes the form of a negative electrode. And in yet other particular embodiments, the drug electrode assembly 200a-d is specifically configured to deliver a drug species when the electrode 202a-d alternatively switches between taking the form of a positive and a negative electrode, during drug delivery. The assist ion source and the assist ion sink components for each of the drug electrode assembly embodiments depicted in FIGS. 2A-2D are identified as such in the table, below, and this depends on the polarity of the electrode.

| Drug | | | | | | |
|---|---|---|---|---|---|---|
| Electrode | Positive Electrode | | Negative Electrode | | Alternating Polarity | |
| Assembly | Sink | Source | Sink | Source | Sink | Source |
| 200a | Electrode | Drug | Drug | Electrode | Electrode | Drug |
| | 202a | Reservoir | Reservoir | 202a | (+) | Reservoir |
| | | 208 | 208 | | | (+) |
| | | | | | Drug | Electrode |
| | | | | | Reservoir | (−) |
| | | | | | (−) | |
| 200b | Electrode | Drug | Drug | Electrode | Electrode | Drug |
| | 202b | Reservoir | Reservoir | 202b | (+) | Reservoir |
| | | 208 | 208 | | | (+) |
| | | | | | Drug | Electrode |
| | | | | | Reservoir | (−) |
| | | | | | (−) | |
| 200c | Interlayer | Drug | Drug | Interlayer | Interlayer | Drug |
| | 202c | Reservoir | Reservoir | 202c | (+) | Reservoir |
| | | 208 | 208 | | | (+) |
| | | | | | Drug | Interlayer |
| | | | | | Reservoir | (−) |
| | | | | | (−) | |
| 200d | Interlayer | Drug | Drug | Interlayer | Interlayer | Drug |
| | 202d | Reservoir | Reservoir | 202d | (+) | Reservoir |
| | | 208 | 208 | | | (+) |
| | | | | | Drug | Interlayer |
| | | | | | Reservoir | (−) |
| | | | | | (−) | |

Electrotransport Drug Delivery Devices

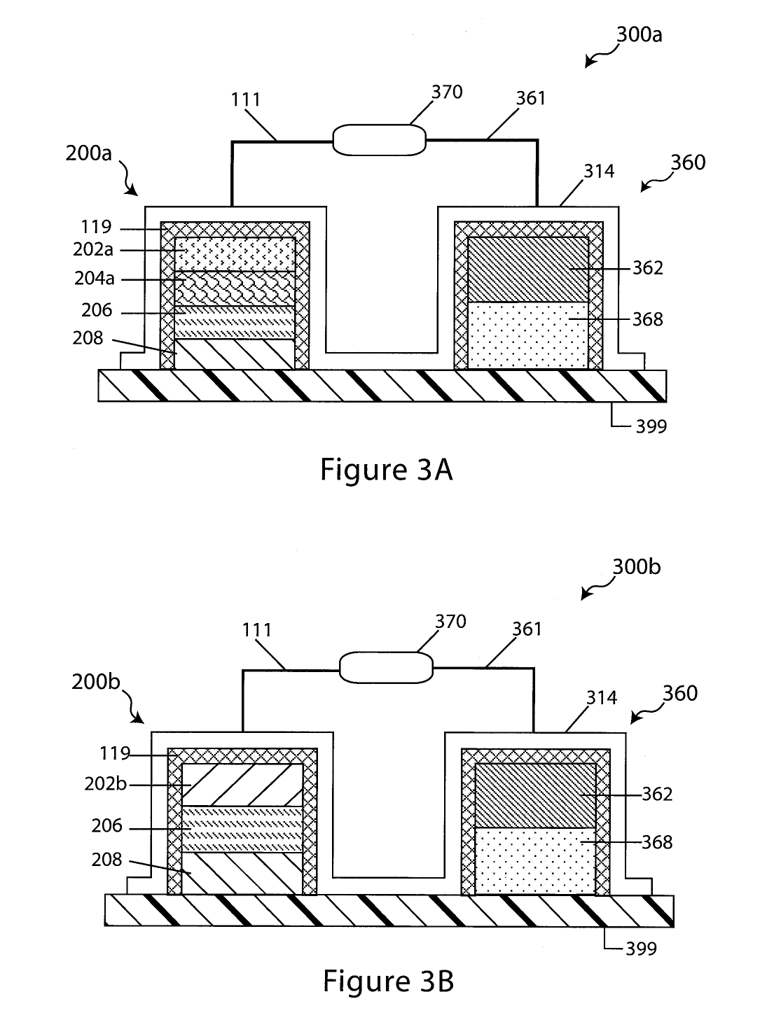

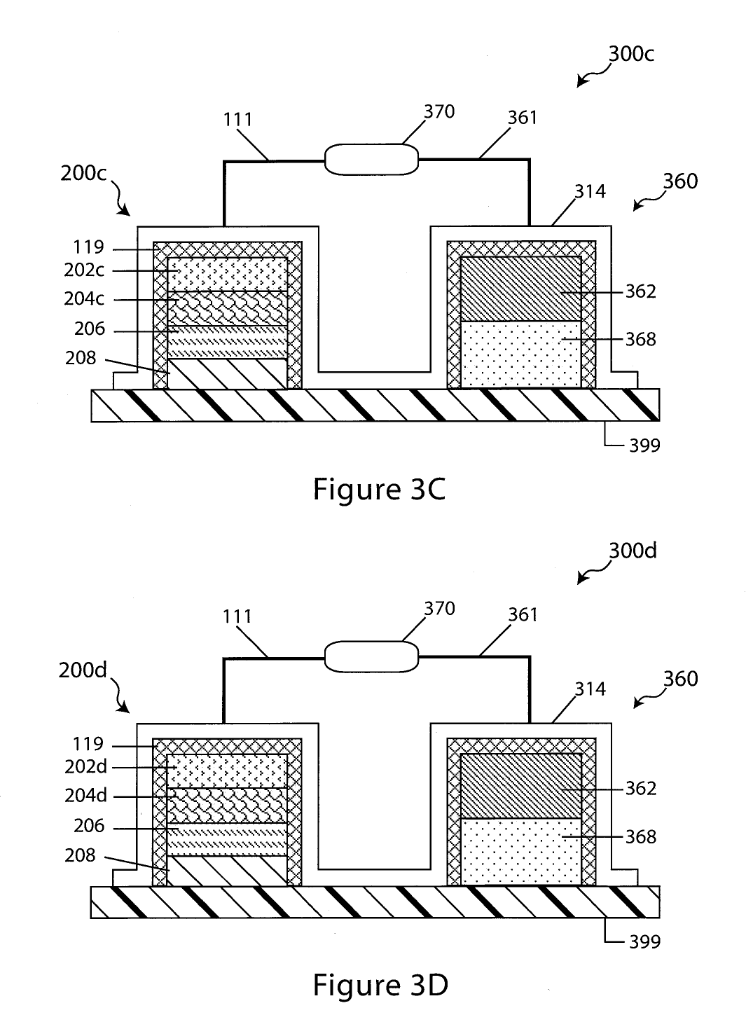

The invention is now described with reference to FIGS. 3A-3D which illustrates, schematically, in cross section, four embodiments of an electrotransport drug delivery device in accordance with the instant invention for delivering (e.g., transdermally) one or more drug species to a biological surface (e.g., a tissue surface such as skin) of a subject for which administration of the drug is intended. With reference to FIGS. 3A-3D, the device (300a-d) has a first electrode assembly (200a-d) and a second electrode assembly 360. The first electrode assembly (200a-d) depicted therein is a drug electrode assembly that is the same as that depicted in FIGS. 2A-2D (i.e., with Na⁺ as the assist ion), and similar elements are similarly numbered. The second electrode assembly 360 is an indifferent electrode assembly 360, which completes the electrical circuit through the tissue 399.

The indifferent electrode assembly 360 generally comprises an indifferent electrode 362 having a first surface and an electrolyte reservoir 368 in contact with and substantially covering that electrode first surface and the reservoir in contact with the tissue surface. The indifferent assembly completes the electrical circuit through the tissue and is generally chosen such that the indifferent electrode itself and any products of electro⁻reduction or electro⁻oxidation are innocuous to the subject being treated. The indifferent assembly also comprises an exterior electrical connector 361 that is electrically coupled to the indifferent electrode. The indifferent electrode may be any suitable iontophoretic electrode as described, for example, in U.S. Pat. No. 5,405,317 and/or U.S. Pat. No. 5,135,477 which are incorporated herein by reference for all that they contain.

The indifferent electrode may also be chosen for its ability to provide a galvanic couple in combination with the electrode 200a-d of the drug assembly and to provide the electromotive driving force for the electrochemical reactions and to drive the Na⁺ ion current across the barrier layer and the drug across the tissue surface. The galvanic couple can also be used to provide electrical power for any optional device control circuitry. Thus, while certain embodiments, contemplate the use of a galvanic couple to drive drug delivery without the use of an external power supply, the invention is not limited as such and also contemplates the use of an external power supply, such as a battery, to assist in driving the current and/or powering peripheral electronics.

By way of illustration the indifferent electrode can be an inert electrode (e.g., a metal foil such as stainless steel), or more commonly a reactive electrode (e.g., Ag/AgCl electrode). In certain embodiments the indifferent electrode 362 is a reactive electrode that generally comprises a metallic salt in contact with a metal. For example silver chloride in contact with metallic silver (Ag/AgCl electrode) or iron chloride in contact with metallic iron (Fe/FeCl electrode). In certain embodiments, the indifferent electrode is a Ag/AgCl electrode such as is known to those of skill in the art of ionotophoretic drug delivery. In such instances, the supporting electrolyte generally contains a sodium chloride salt with a suitable buffer (e.g., sodium phosphate buffer). During device operation, the Ag/AgCl electrode may be electrochemically reduced or oxidized, as AgCl on the surface of the metallic silver electrode is reduced to give silver metal and chloride anion or when the electrode is oxidized silver goes to silver ions as follows:

AgCl+e⁻ [symbol] Ag+Cl⁻

Ag−e⁻ [symbol] Ag⁺

The indifferent electrolyte reservoir is generally composed of a polymer gel matrix or polymer gel (e.g., hydrogel) having a supporting electrolyte solution employed in the electrolyte reservoir, the type of electrolyte solution depends, in part, on the type of indifferent electrode employed. Supporting electrolyte solutions that are suitable for indifferent electrodes useful for the instant invention are known in the art of iontophoretic drug delivery. Generally they are pharmacologically non⁻toxic and chemically inert. Suitable salts include, but are not limited to, sodium chloride, sulfates, nitrates, phosphates, citrates and mixtures thereof. The addition of a buffer is also useful. For example, when the indifferent electrode is a Ag/AgCl electrode the electrolyte solution can be an aqueous solution containing sodium chloride (e.g., a 0.1 molar salt solution).

The drug electrode assembly 200a-d and the indifferent assembly 360 are electrically coupled, via their respective exterior connectors 111 and 361, to an electronic control/power source unit 370, which can be used to control current and adjust drug delivery rate as well as provide power, for instance by means of a battery, to drive the electrical current of the device and to power device electronics. The electrical circuitry may be as simple as a single precision resistor selected for a desired rate of drug delivery, or a set of resistors that can be toggled over the course of drug delivery to control the rate as a function of dose and time. The control unit can include a microprocessor to control current through the device in a pre⁻programmed fashion as a function of time. Such electrical components can be utilized to regulate the level, waveform, timing and other aspects of the electrical current and/or to adapt the current over time or in response to changes in conductivity of the tissue and/or device. Such electrical circuits are well known to those of skill in the art and are described, for example, in U.S. Pat. No. 5,533,971.

During drug delivery an external electrical current flows between the drug electrode assembly and the second assembly, e.g., the indifferent assembly. The external electrical current is so called because electrons flow between the drug electrode assembly and the indifferent electrode assembly via their respective exterior connectors and across the electronic control/power supply unit, which is exterior to the assembly innards and generally exterior to the assembly housing. When electrons of the external electrical current flow into an electrode assembly, that assembly is a positive assembly, and when electrons flow out of an electrode assembly that assembly is a negative assembly.

Also illustrated is a housing support structure 314 (e.g., flexible polymer), and an optional means 316 of affixing the device to a skin surface 299 using, e.g., a biocompatible pressure sensitive adhesive. An electrical insulator generally separates the first assembly from the second assembly; for instance, as shown, the housing support structure or an integrated component thereof, or an air gap, or a non⁻conductive polymer (e.g., polyethylene spacer or like) could provide this separation. In operation, the device is arranged such that both the first and the second electrode assembly are configured for ion transport to a portion of tissue of a subject for which drug delivery is intended.

In certain particular embodiments, the electrotransport device depicted in FIGS. 3A-3D is configured to the skin surface of a subject for delivery of an anionic drug. The anionic drug can be administered to the subject through the skin surface by supplying an external electrical current between the drug electrode assembly 200a-d and the indifferent electrode assembly 360, which is derived from the power supply 370. When administering the drug, anionic drug species move to the tissue surface as the drug electrode assembly takes the form of a positive assembly and the indifferent assembly negative.

In certain particular embodiments, the electrotransport device depicted in FIGS. 3A-3D is configured to the skin surface of a subject for delivery of a charge neutral drug species, when the electrode of the drug electrode assembly takes the form of a negative electrode. The charge neutral drug species can be administered to the subject through the skin surface by supplying an external electrical current between the drug electrode assembly 200a-d and the indifferent electrode assembly 360, which is derived from the power supply 370. When administering the drug, neutral drug species move to the tissue surface as the drug electrode assembly takes the form of a negative assembly and the indifferent assembly positive.

In certain particular embodiments, the electrotransport device depicted in FIGS. 3A-3D, is configured to the skin surface of a subject for delivery of a neutral drug species, when the electrode of the drug electrode assembly alternatively switches between taking the form of a negative electrode and a positive electrode. The charge neutral drug species can be administered to the subject through the skin surface by supplying an external alternating electrical current between the drug electrode assembly 200a-d and the indifferent electrode assembly 360, which is derived from the power supply 370.

In certain particular embodiments, the electrotransport device depicted in FIGS. 3A-3D, is configured to the skin surface of a subject for delivery of an anionic drug species, when the electrode of the drug electrode assembly alternatively switches between taking the form of a negative electrode and a positive electrode. The anionic drug species can be administered to the subject through the skin surface by supplying an external alternating electrical current between the drug electrode assembly 200a-d and the indifferent electrode assembly 360, which is derived from the power supply 370.

### Illustrative Embodiments

Anionic Drug Delivery (Using a Sodium Reactive Electrode)

In one embodiment, the electrotransport device depicted in FIG. 3A and described above is configured to the skin surface of a subject for delivery of an anionic drug. The assist ion of the drug electrode assembly is sodium ions (Na⁺ ions)

By way of illustration, the device 300a comprises a drug electrode assembly 200a comprising a sodium reactive electrode 202a, e.g., a sodium intercalation electrode comprising a MnO₂ intercalation host material; a barrier layer 206, e.g., a sintered Nasicon or beta-alumina monolith; an interlayer 204a of a non-aqueous liquid organic electrolyte solution, e.g., a 1 molar sodium salt dissolved in an aprotic liquid organic solvent; a drug reservoir 208, e.g., a gel of an aqueous or non-aquoeus solution of a sodium drug salt, the drug anion existing as an ion in the electrolyte solution; and an indifferent electrode assembly 360, e.g., an indifferent AgAgCl assembly as described.

The anionic drug can be administered to the subject through the skin surface by supplying a external electrical current between the drug electrode assembly 200a and the indifferent electrode assembly 360, which is derived from the power supply 370. When administering the drug, anionic drug species move to the tissue surface as the drug electrode assembly takes the form of a positive assembly and the indifferent assembly negative. The electrochemical reactions at the positive (1) and negative assembly (2) is in accordance with the following electrochemical reaction:

MnO₂+e⁻+xNa⁺ [symbol] NaₓMnO₂ (Drug Electrode Assembly) (1)

Ag−e⁻ [symbol] Ag⁺(Indifferent Electrode Assembly) (2)

Neutral Drug Delivery (Driven by a Galvanic Potential Difference)

In another embodiment, the electrotransport device 300a depicted in FIG. 3A can be usefully employed for the delivery of a neutrally charged drug, and by proper selection of the electrodes, a significant open circuit galvanic potential can be generated between the electrode 200a and the indifferent electrode 362. The assist ion of the drug electrode assembly is sodium ions (Na⁺ ions).

The device 300a comprising a drug electrode assembly 208 comprising a sodium reactive electrode 200a, e.g., sodium metal foil; a barrier layer 206, e.g., a sintered Nasicon or beta-alumina monolith; an interlayer 204a of a non⁻aqueous liquid organic electrolyte solution, e.g., a 1 molar sodium salt dissolved in an aprotic liquid organic solvent; a drug reservoir 208, e.g., a gel of an aqueous or non-aqueous solution of a NaCl salt, the neutral drug dispersed throughout the gel; and an indifferent electrode assembly 360, e.g., an indifferent AgAgCl assembly as described above. An open circuit galvanic potential difference of about 3 Volts is generated between the positive (1) and negative assembly (2) in accordance with the following electrochemical reaction:

Na [symbol] Na⁺+e⁻ (Anode; ⁻2.7 V vs. SHE) (1)

AgCl+e⁻ [symbol] Ag+Cl⁻ (Cathode; 0.23 vs. SHE) (2)

The neutral drug can be administered to the subject through the skin surface by supplying an external electrical current between the drug electrode assembly 200a and the indifferent electrode assembly 360, which is derived from the power supply 370. When administering the drug, neutral drug species move to the tissue surface as the drug electrode assembly takes the form of a negative assembly and the indifferent assembly positive.

Alternatively, the galvanic potential difference can be used to fully or partially drive the device current and cause the drug to move to the tissue surface. For instance, by activating a switch in the electronic control unit 370 that activates the external electrical current to flow, the current driven fully or in part by the galvanic potential difference between the drug assembly and the indifferent assembly.
Neutral Drug Delivery (Using Alternating Current Between Two Sodium Electrodes)

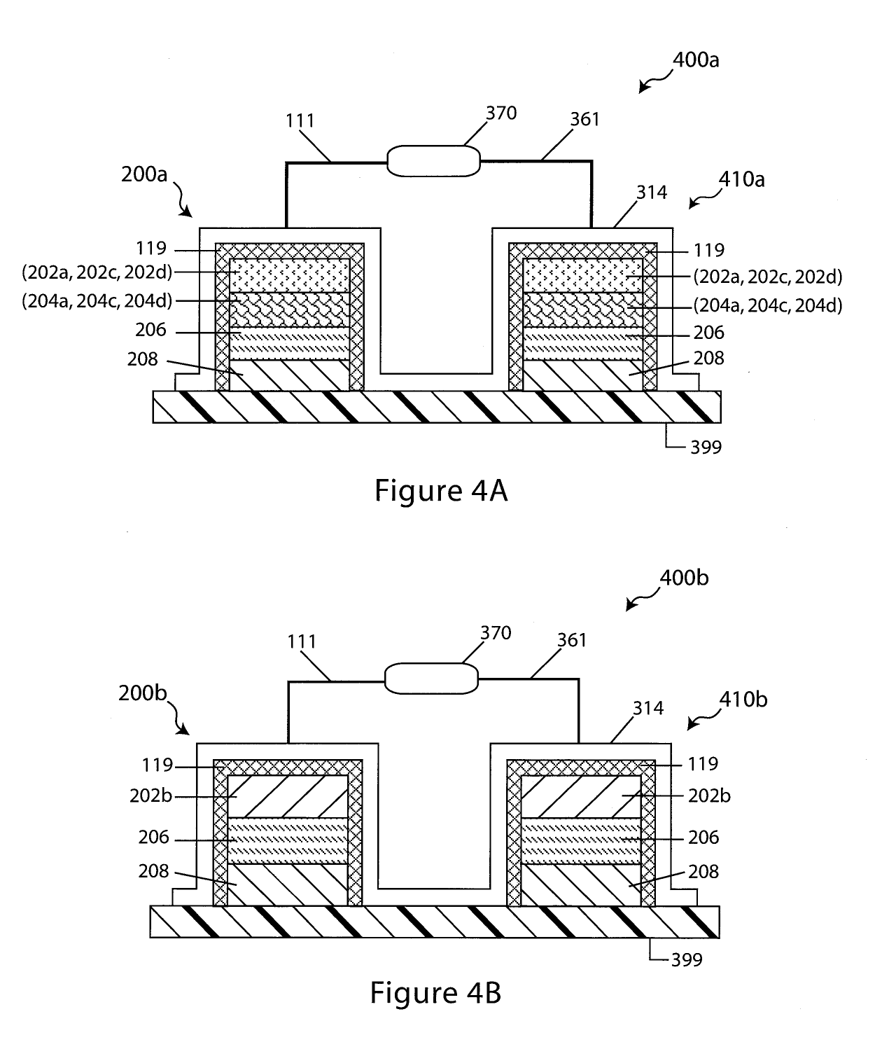

In yet another embodiment, the electrotransport device 400b depicted in FIG. 4B can be usefully employed for the delivery of a neutrally charged drug. The assist ion of the drug electrode assembly is sodium ions (Na⁺ ions). The device 400b comprises a first drug electrode assembly 200b and a second drug electrode assembly 410b. The first drug electrode assembly 200b is an assembly as depicted in FIG. 2B, and the second drug electrode assembly 410b is substantially the same as the first assembly 200b, and similar elements are similarly numbered so a description of the first assembly is sufficient for a description of both. In an alternative embodiment the second assembly can be different than the first assembly, or they may have similar architecture but different elements.

By way of illustration, electrode 200b can be an inorganic solid⁻state sodium reactive electrode, e.g., a sodium metal alloy deposited by thermal evaporation onto the first surface of barrier layer 206, e.g., a beta-alumina sintered monolith, and drug reservoir 208, e.g., a gel of an aqueous or non-aqueous solution of a NaCl salt, the neutral drug dispersed throughout the hydrogel.

The neutral drug can be administered to the subject through the skin surface by supplying an external alternating electrical current between the first drug electrode assembly 200b and the second drug electrode assembly 410b, which is derived from the power supply 370. When electrons of the external current flow into the first drug electrode assembly, that assembly takes the form of a positive assembly and the second assembly is negative, and when the alternating current changes direction, the electrodes respectively switch their polarity.
Anionic Drug Delivery (Using an Improved Ag/AgCl Drug Electrode Assembly)

In another embodiment, the electrotransport device 300c depicted in FIG. 3C can be usefully employed for the delivery of an anionic drug. The device 300c comprises a drug electrode assembly 200c and an indifferent electrode assembly 360. The assist ion of the drug electrode assembly 200c is sodium ions (Na⁺ ions). The first drug electrode assembly 200c is an assembly as depicted in FIG. 2C and the indifferent electrode assembly 360 is an indifferent assembly as depicted in FIG. 3C, and similar elements are similarly numbered.

By way of illustration, the device 300c comprises a drug electrode assembly comprising an electrode 202c, e.g., Ag/AgCl electrode, an interlayer 204c, e.g., an aqueous solution of a metal salt (e.g., NaCl); a barrier layer 206, e.g., a beta-alumina of Nasicon monolith; and a drug reservoir 208, e.g., a gel of an aqueous or non-aqueous solution of a sodium drug salt, and an indifferent electrode assembly 360, e.g., a Ag/AgCl indifferent assembly as described above.

The electrochemical reactions at the two electrodes are considered to be as follows:

AgCl+e⁻ [symbol] Ag+Cl⁻ (Positive Drug Electrode Assembly) (1)

Ag−e⁻ [symbol] Ag⁺ (Negative Indifferent Electrode Assembly) (2)

The anionic drug can be administered to the subject through the skin surface by supplying an external electrical current between the drug electrode assembly 200c and the indifferent electrode assembly 360, which is derived from the power supply 370. When administering the drug, anionic drug species move to the tissue surface as the drug electrode assembly takes the form of a positive assembly and the indifferent assembly negative.

### Other Embodiments

In accordance with the principles described above for a drug electrode assembly wherein the assist ion of the assembly is Na⁺ ions, and electrotransport devices and apparatus' thereof, certain other embodiments in accordance with the present invention will be apparent to one of skill in the art including a drug electrode assembly wherein the assist ion is an other species of alkali metal ions, such as Li⁺ ions or K⁺ ions.

Suitable inorganic solid-state Li⁺ ion conductors which can be fabricated into a barrier layer in accordance with the principles described above include, but are not limited to inorganic glassy or amorphous lithium ion conductors, such as, but not limited to lithium ion conducting silicate glasses having appropriate modifiers and network formers.

Suitable ceramic and glass-ceramic Li⁺ ion conducting solid⁻state electrolyte materials which can be fabricated into a barrier layer in accordance with the principles described above include, but are not limited to, lithium metal phosphates such as those of the nasicon type (e.g., Li_{1.3}Ti_{1.7}Al_{0.3}(PO₄)₃) and the like. Suitable ceramic solid⁻state electrolyte materials include lithium metal oxides such as those of the perovskite type (e.g., (Li,La)TiO₃ and those of the garnet type (e.g., Li₅La₃M₂O₁₂ (M=Nb, Ta), and lithium beta alumina; and the like.

For instance, ceramics and glass-ceramics suitable as a Li⁺ ion conducting solid⁻state electrolyte material include lithium metal phosphates such as lithium titanium phosphates, lithium germanium phosphates and lithium hafnium phosphates and combinations thereof, and for example prepared by processes such as, but not limited to, calcination and melt/quenching. For instance those of the type LiM₂(PO₄)₃, M=Ge, Ti, Sn, Hf, Zr, and the like. For example Li₁⁺ₓMₓ(Ti, Ge, Hf)₂⁻ₓ(PO₄)₃ where M is an element selected from the group consisting of Fe, Ga, Al and rare earth elements and where 0.1≦x≦1.9; such as, for example where x is about 0.3. For example, Li₁⁺ₓ⁺_{y}(Al,Ga)ₓ(Ti, Ge, Hf)₂⁻ₓSi_{y}P₃⁻_{y}O₁₂ where 0.1≦x≦1 and 0.1≦y≦1; such as, Li₁⁺ₓ⁺_{y}(Al,Ga)ₓ(Ti, Ge)₂⁻ₓSi_{y}P₃⁻_{y}O₁₂ where 0.1≦x≦1 and 0.1≦y≦1; and Li₁⁺ₓ⁺_{y}AlₓTi₂⁻ₓSi_{y}P₃⁻_{y}O₁₂ where 0.1≦x≦1 and 0.1≦y≦1. For example, Li₁⁺ₓ⁺_{y}AlₓTi₂⁻ₓSi_{y}P₃⁻_{y}O₁₂ where 0.1≦x≦0.3 and 0.1≦y≦0.4 shows excellent conductivity.

Other specific examples of ceramics and glass⁻ceramics suitable as a Li⁺ ion conducting solid-state electrolyte material include Li_{0.3}La_{0.5}TiO₃, Li₂O.11Al₂O₃, Li₅La₃Ta₂O₁₂, Li₅La₃Nb₂O₁₂, Li₅TiP₃O₁₂, Li₃Fe₂P₃O₁₂, Li₄NbP₃O₁₂, Li₅ZrP₃O₁₂, Li₁₄Zn(GeO₄)₄, Li₄NbP₃O₁₂, Li₃Zr₂Si₂PO₁₂, Li₃Zr₂Si₂PO₁₂

Suitable ceramic and glass ceramic Li⁺ ion conductors useful as an impervious solid⁻state electrolyte material are described, for example in U.S. Pat. No. 4,985,317, and U.S. Patent Application Pub. No. 2007/0087269 which is incorporated by reference herein in its entirety and for all purposes.

One particularly suitable impervious solid⁻state electrolyte material for use is a glass⁻ceramic of the following composition:

| Composition | | Mol % |
|---|---|---|
| P₂O₅ | | 26-55% |
| SiO₂ | | 0-15% |
| GeO₂ ⁺ TiO₂ | | 25-50% |
| In which | GeO₂ | 0-50% |
| | TiO₂ | 0-50% |
| ZrO₂ | | 0-10% |
| M₂O₃ | | 0-10% |
| Al₂O₃ | | 0-15% |
| Ga₂O₃ | | 0-15% |
| Li₂O | | 3-25% |

and containing a predominant crystalline phase composed of Li₁⁺ₓ(M,Al,Ga)ₓ(Ge₁⁻_{y}Ti_{y})₂⁻ₓ(PO₄)₃ where X≦0.8 and 0≦Y≦1.0, and where M is an element selected from the group consisting of Nd, Sm, Eu, Gd, Tb, Dy, Ho, Er, Tm and Yb and/or Li₁⁺ₓ⁺_{y}QₓTi₂⁻ₓSi_{y}P₃⁻_{y}O₁₂ where 0<X≦0.4 and 0<Y≦0.6, and where Q is Al or Ga.

The glass-ceramics are obtained by melting raw materials to a melt, casting the melt to a glass and subjecting the glass to a heat treatment. Such materials are available from OHARA Corporation, Japan and are further described in U.S. Pat. Nos. 5,702,995, 6,030,909, 6,315,881 and 6,485,622, which are incorporated herein by reference.

Suitable Li⁺ ion conducting membranes which are suitable for use as a Li⁺ ion conducting barrier layer in accordance with principles described herein are further described in the following U.S. Patents and these are incorporated herein by reference. Freestanding layers consistent with the principles described above for a barrier layer and which can be usefully employed as a barrier layer in protective architectures in accordance with the instant invention are disclosed in the following US Patents and Patent Applications, all of which are hereby incorporated by reference herein: i) suitable barrier layers are described in U.S. Patent Application Pub. No. US 2007/0087269 to Inda, where the barrier layer is generally referred to as a solid electrolyte sheet which is made by sintering an inorganic substance powder by first fabricating a greensheet comprising the inorganic substance powder followed by sintering; ii) suitable barrier layers are described in U.S. Pat. No. 4,985,317 to Adachi where the barrier layer is generally referred to as a solid electrolyte formed by sintering and solid electrolyte sheets; iii) suitable barrier layers are described in U.S. Patent Application Pub. No. US 2007/0117026 to Kumar where the barrier layer is generally referred to as a sintered membrane and composite membrane fabricated by tape⁻casting followed by sintering of a glass or glass⁻ceramic powder; and iv) particularly suitable barrier layers are described in U.S. Pat. Nos. U.S. Pat. No. 5,02,995; U.S. Pat. No. 6,030,909; U.S. Pat. No. 6,315,881; and U.S. Pat. No. 6,485,622 to Fu and assigned to Kabushiki Kaisha Ohara, where the barrier layer is generally referred to as a glass⁻ceramic layer fabricated by glass⁻ceramic processing. Glass⁻ceramic layers as described above in the Fu references, are generally available from the Ohara Corporation.

Lithium electroactive component materials suitable for use as or in a lithium reactive electrode in accordance with the principles described herein for a sodium reactive electrode include lithium metal, lithium metal alloys and lithium intercalation materials. Intercalation compounds suitable for use as the electroactive material include, but are not limited to, lithium metal chalcogenides (e.g., oxides), lithium metal phosphates, and lithium metal silicates and various carbons capable of intercalating Li ions—especially, lithium transition metal oxides, phosphates and silicates. Specific examples include LiₓC6, graphites, LiNiO₂, Li₄Ti₅O₁₂, LiMn₂O₄, LiCoO₂, LiNiₓCo₁⁻ₓO₂, LiFePO₄, LiFe₃(PO₄)₃, LiC₆, LiWO₂ and LiMoO₂.

Suitable inorganic solid⁻state K⁺ion conductors which can be fabricated into a barrier layer in accordance with the principles described above include, but are not limited to potassium beta alumina materials generally including potassium β-Al₂O₃ and β″-Al₂O₃ e.g., K₂O0.5Al₂O₃, K₂O.11Al₂O₃ K₂O.xAl₂O₃ where 8≦X≦11.

Potassium electroactive component materials suitable for use as or in a potassium reactive electrode in accordance with the principles described herein for a sodium reactive electrode include potassium metal, potassium metal alloys and potassium intercalation materials. Intercalation compounds suitable for use as the electroactive material include, but are not limited to, potassium metal chalcogenides (e.g., oxides), potassium metal phosphates, and potassium metal silicates and various carbons capable of intercalating K⁺ ions—especially, potassium transition metal oxides, phosphates and silicates.

In accordance with the principles described above for a drug electrode assembly wherein the assist ion of the assembly is Na⁺ ions, and electrotransport devices and apparatus' thereof, certain other embodiments in accordance with the present invention will be apparent to one of skill in the art including a drug electrode assembly wherein the assist ion is Ag⁺ ions or Cu⁺ or Cu⁺ ions.

Suitable inorganic solid⁻state Ag⁺ and Cu⁺ ion conductors which can be fabricated into a barrier layer in accordance with the principles described above include, but are not limited to Ag4RbI₅, Ag-β″ alumina and Cu₁₆Rb₄I₇Cl₁₃, depending on whether the “assist-cation” is Ag or Cu.

In accordance with the principles described above for a drug electrode assembly wherein the assist ion of the assembly is Na⁺ ions, and electrotransport devices and apparatus' thereof, certain other embodiments in accordance with the present invention will be apparent to one of skill in the art including a drug electrode assembly wherein the assist ion is Cl⁻ ions or F⁻ ions.

Suitable inorganic solid-state F⁻ ion conductors suitable for the fabrication of a F⁻ ion conducting barrier-layer include LaF₃ and PbF₂.

Device Implementation

Electro-transport devices in accordance with the present invention may be implemented as topically applied devices (e.g., patches), as would be understood by those of skill in the art given the parameters provided herein. The arrangement between the electrodes can take on any number of suitable formats. In one embodiment, the electrodes are placed adjacent to each other in ionic communication with the skin in a side-by-side configuration separated by an air gap or insulating material. Generally, the electro-transport device will comprise a housing support structure made of a non-conductive material preferably made of a polymeric material that can be rigid, but is preferably flexible. The device can further include a means for affixing the device to a tissue (e.g., a skin) surface. Various means are known to those of skill in the art. One such approach utilizes a bio-compatible adhesive (e.g., polyisobutylene) around the periphery of the device to keep it attached to the body surface. Such adhesives are well known in the art of iontophoretic drug delivery systems.

In one embodiment, the donor electrode assembly comprises a stand-alone anode patch that can be incorporated into a drug delivery device by connecting it to a corresponding second electrode assembly patch or inserting it into an electro-transport device structure. In another illustrative embodiment, the donor and second electrode assemblies can be aligned adjacent to each other in a concentric ring fashion such that the second electrode is adjacent to the donor electrode and surrounds it around its outer periphery. The donor electrode is of circular geometry. A spacer is placed between the donor electrode and the second electrode, such as that described herein as an air gap or an insulating material. Alternatively, the donor and second electrode may also exchange positions. Moreover, the geometry is not limited to a circular embodiment, but includes other geometries such as rectangular and oval.

The foregoing embodiments are intended to be illustrative and not limiting. Using the teaching provided herein, other electrode arrangements will be available to one of skill.

Kits

In another embodiment, this invention provides kits for delivering a drug to a mammal. The kits typically comprise an electro-transport device as described herein. In certain embodiments the electro-transport device can be packaged in a container and/or can have removable protective caps or film, or other barrier over one or both electrodes that can readily be removed before use. Optionally, the kits can additionally contain an electrode cream, gel, ointment, fluid, or paste (e.g., a skin or other tissue compatible conductive medium) to promote good electrical contact (ion communication) between one or both electrodes and the surface to which the device is to be applied. Optionally, the cream, gel, ointment, fluid or paste can be provided already applied to the electrode surface(s) of the device. Optionally the kits can also include means (e.g., a solvent impregnated wipe or swab) for cleaning and/or disinfecting a tissue surface prior to application of the device. Optionally, the device can also include means (other than those that may be present on the device itself) for affixing the device to a tissue surface. Such means include, but are not limited to liquid, gel, paste adhesive and/or adhesive strips, and the like.

The kit can, optionally, further comprise one or more other agents typically administered to a subject being administered a particular drug. In addition, the kits optionally include labeling and/or instructional materials providing directions (i.e., protocols) for the use of the devices described herein. In certain embodiments preferred instructional materials describe use the devices described herein for administering a drug to a subject in need thereof. The instructions optionally teach methods of applying the device to the subject, and/or methods of calibrating or adjusting the device to calibrate or adjust the rate of drug delivery. The instructional materials may also, optionally, teach preferred dosages/therapeutic regimen, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

Conclusion

Drug electrode assemblies, electro-transport devices and methods for the delivery of anionic drugs, cationic drugs and neutrally charged drug molecules through a body surface (e.g., transdermally through the skin) by electro-transport are described herein.

Although the foregoing invention has been described in some detail for purposes of clarity of understanding, it will be apparent that certain changes and modifications may be practiced within the scope of the appended claims. It should be noted that there are many alternative ways of implementing both the process and compositions of the present invention. Accordingly, the present embodiments are to be considered as illustrative and not restrictive, and the invention is not to be limited to the details given herein.

1. A drug electrode assembly for electrotransport delivery of a drug across a tissue surface of a subject, the assembly comprising:
an electrode having a first surface;
a drug reservoir comprising a drug species and an electrolyte solution conductive to Na⁺ ions; and
a liquid impermeable solid-state Na⁺ ion conducting barrier layer, the barrier layer having a first surface and second surface, the first surface of the barrier layer facing the electrode and the second surface of the barrier layer facing the drug reservoir.

2. The drug electrode assembly of claim 1, wherein the electrolyte solution of the drug reservoir physically contacts and substantially covers the barrier layer second surface, and the barrier layer is impermeable to the electrolyte solution and the solvent molecules of the electrolyte solution.

3. The drug electrode assembly of claim 2, wherein the electrolyte solution of the drug reservoir is aqueous, and the barrier layer is impermeable to water molecules.

4. The drug electrode assembly of claim 2, wherein the drug reservoir comprises water and the barrier layer is impermeable to water molecules.

5. The drug electrode assembly of claim 1, further comprising an interlayer interposed between the barrier layer and the electrode, the interlayer comprising a Na⁺ ion conducting electrolyte solution physically contacting the first surface of the electrode and physically contacting the first surface of the barrier layer, and wherein the barrier layer is impermeable to the electrolyte solution of the interlayer and its solvent molecules.

6. The drug electrode assembly of claim 5, wherein the electrolyte solution of the interlayer is a non-aqueous organic electrolyte comprising an organic liquid, the barrier layer impermeable to the molecules of that organic liquid.

7. The drug electrode assembly of claim 6, wherein the drug reservoir comprises water, the barrier layer impermeable to both water molecules and the molecules of the organic liquid.

8. The drug electrode assembly of claim 1, wherein the barrier layer is impermeable to fluids selected from the group consisting of water vapor, oxygen and carbon dioxide.

9. The drug electrode assembly of claim 3, wherein the barrier layer is impervious to the aqueous electrolyte solution of the drug reservoir and impervious to water molecules.

10. The drug electrode assembly of claim 6, wherein the barrier layer is impervious to the organic liquid and impervious to molecules of the organic liquid.

11. The drug electrode assembly of claim 4, wherein the barrier layer is impervious to water molecules.

12. The drug electrode assembly of claim 1, wherein the barrier layer is dry.

13. The drug electrode assembly of claim 1, wherein the barrier layer is a single⁻ion conductor of Na⁺ ions.

14. The drug electrode assembly of claim 1, wherein the barrier layer is only permeable to Na⁺ ions.

15. The drug electrode assembly of claim 1, wherein the barrier layer comprises an inorganic solid-state conductive medium that provides a continuous solid-state pathway for electrical migration of Na⁺ ions across the barrier layer, and the solid-state conductive medium is a single⁻ion conductor of Na⁺ ions.

16. The drug electrode assembly of claim 15, wherein the solid state conductive medium comprises a solid⁻state electrolyte material selected from the group consisting of Nasicon (sodium super ion conductor) materials generally, including Na₁₊ₓZrₓP₃₋ₓSiₓO₁₂ where O≦X≦3, Na₃M₂(PO₄)₃ where M=Sc, Cr or Fe, Na₅RESi₄O₁₂ where RE is Yttrium or any rare earth metal, Na₅SmSi₄O₁₂, Na₅DySi₄O₁₂, Na₃Zr₂Si₂PO₁₂, Na₅GdSi₄O₁₂, NaxTi₃P₆Si₂O₂₅ where 0≦X≦2 and beta alumina materials generally including B—Al₂O₃, B″—Al₂O₃, Na₂O0.5Al₂O₃, Na₂O.11Al₂O₃Na₂O.xAl₂O₃ where 8≦X≦11.

17. The drug electrode assembly of claim 1, wherein the barrier layer is an inorganic and dense monolithic layer, having a porosity of less than 10% by volume.

18. The drug electrode assembly of claim 1, wherein the barrier layer is a multilayer composite laminate comprising at least two layers, a first assist ion conducting layer forming the first surface of the barrier layer, the first layer comprising an impervious solid-state electrolyte material, and a second assist ion conducting layer forming the second surface of the barrier layer, the second layer comprising an impervious solid-state electrolyte material.

19. The drug electrode assembly of claim 18, wherein the impervious solid-state electrolyte material of the first layer is an assist ion conducting inorganic glass and the impervious solid-state electrolyte material of the second layer is an assist ion conducting inorganic polycrystalline ceramic material.

20. The drug electrode assembly of claim 18, wherein the impervious solid-state electrolyte material of the first layer is an assist ion conducting inorganic polycrystalline ceramic and the impervious solid-state electrolyte material of the second layer is an assist ion conducting inorganic glass material.

21. The drug electrode assembly of claim 18, wherein the impervious solid-state electrolyte material of the first layer is an assist ion conducting inorganic polycrystalline ceramic and the impervious solid-state electrolyte material of the second layer is an assist ion conducting inorganic polycrystalline ceramic different than that of the first layer.

22. The drug electrode assembly of claim 21, wherein the solid-state electrolyte material of the first layer is beta alumina and the solid-state electrolyte material of the second layer is Nasicon.

23. The drug electrode assembly of claim 18, wherein the multilayer composite laminate comprises a third layer interposed between the first and second layer.

24. The drug electrode assembly of claim 23, wherein the third layer comprises an impervious solid-state electrolyte material which is an assist ion conducting polycrystalline ceramic and the solid state electrolyte material of the first layer is an assist ion conducting glass and the solid-state electrolyte material of the second layer is an assist ion conducting glass.

25. The drug electrode assembly of claim 1, wherein the electrode is chemically incompatible in contact with water.

26. The drug electrode assembly of claim 1, wherein the electrode is a reactive sodium electrode comprising a sodium electroactive component material.

27. The drug electrode assembly of claim 26, wherein the sodium electroactive component material is sodium metal.

28. The drug electrode assembly of claim 26, wherein the sodium electroactive component material is a sodium metal alloy.

29. An electrotransport device comprising a drug electrode assembly electrically coupled to a second electrode assembly, the drug electrode assembly comprising, an electrode having a first surface;
a drug reservoir comprising a drug species and an electrolyte solution conductive to Na⁺ ions; and
a liquid impermeable solid⁻state Na⁺ ion conducting barrier layer, the barrier layer having a first surface and second surface, the first surface of the barrier layer facing the electrode and the second surface of the barrier layer facing the drug reservoir.

30. The electrotransport device of claim 29, wherein the second assembly is an indifferent electrode assembly.

31. The electrotransport device of claim 29, wherein the second assembly is a second drug electrode assembly.

32. The electrotransport device of claim 29, wherein the second drug electrode assembly is substantially the same as the first drug assembly.

33. The electrotransport device of claim 29, wherein the galvanic potential between the drug assembly and the second assembly is at least 2 Volts.

34. The electrotransport device of claim 29, wherein the galvanic potential between the drug assembly and the second assembly is at least 3 Volts.

35. A method for electrotransport delivery of a drug species across a tissue surface of a subject, the method comprising the steps of:
i) applying to the tissue surface a drug electrode assembly of claim 1;
ii) applying to the tissue surface a second electrode assembly spaced from the first assembly;
iii) supplying an electrical current to the drug assembly and the second assembly to cause the drug species to leave the drug reservoir of the drug assembly and move to the tissue surface of the subject.

36. The method of claim 35, wherein the second assembly is a second drug electrode assembly and the electrical current supplied is an alternating current.

37. A method for electrotransport delivery of a drug species across a tissue surface of a subject, the method comprising the steps of:
i) applying to the tissue surface a drug electrode assembly of claim 1;
ii) applying to the tissue surface a second electrode assembly spaced from the first assembly;
iii) activating a switch that allows an electrical current to flow between the drug assembly and the second assembly and which causes the drug species to leave the drug reservoir of the drug assembly and move to the tissue surface of the subject.

38. A drug electrode assembly for electrotransport delivery of a drug across a tissue surface of a subject, the assembly comprising:
an electrode having a first surface;
a drug reservoir comprising a drug species and an electrolyte solution conductive to assist ions; and
a liquid impermeable solid⁻state assist ion conducting barrier layer, the barrier layer having a first surface and second surface, the first surface of the barrier layer facing the electrode and the second surface of the barrier layer facing the drug reservoir.

39. The drug electrode assembly of claim 38, wherein the assist ion is an alkali metal ion.

40. The drug electrode assembly of claim 38, wherein the assist ion is a halide ion.

41. The drug electrode assembly of claim 38, further comprising an interlayer interposed between the barrier layer and the electrode, the interlayer comprising an assist ion conducting electrolyte solution physically contacting the first surface of the electrode and physically contacting the first surface of the barrier layer, and wherein the barrier layer is impermeable to the electrolyte solution of the interlayer and its solvent molecules.
